SEQUENCE LISTING (76 sequences)

SEQ ID NO: 1 - protein, 36 aa, Homo sapiens
YPIKPEAPGEDASPEELNRYYASLRHYLNLVTRQRY

SEQ ID NO: 2 - protein, 22 aa, Homo sapiens
DELNRYYASLRHYLNLVTRQRY

SEQ ID NO: 3 - protein, 24 aa, Homo sapiens
TPEELNRYYASLRHYLNLVTRQRY

SEQ ID NO: 4 - protein, 25 aa, Homo sapiens
VSPEELNRYYASLRHYLNLVTRQRY

SEQ ID NO: 5 - protein, 26 aa, Homo sapiens
EASPEELNRYYASLRHYLNLVTRQRY

SEQ ID NO: 6 - protein, 27 aa, Homo sapiens
DDASPEELNRYYASLRHYLNLVTRQRY

SEQ ID NO: 7 - protein, 30 aa, Homo sapiens
VPGEDASPEELNRYYASLRHYLNLVTRQRY

SEQ ID NO: 8 - protein, 31 aa, Homo sapiens
DAPGEDASPEELNRYYASLRHYLNLVTRQRY

SEQ ID NO: 9 - protein, 33 aa, Homo sapiens
QPEAPGEDASPEELNRYYASLRHYLNLVTRQRY

SEQ ID NO: 10 - protein, 33 aa, Homo sapiens
RPEAPGEDASPEELNRYYASLRHYLNLVTRQRY

SEQ ID NO: 11 - protein, 33 aa, Homo sapiens
NPEAPGEDASPEELNRYYASLRHYLNLVTRQRY

SEQ ID NO: 12 - protein, 34 aa, Homo sapiens
VKPEAPGEDASPEELNRYYASLRHYLNLVTRQRY

SEQ ID NO: 13 - protein, 34 aa, Homo sapiens
LKPEAPGEDASPEELNRYYASLRHYLNLVTRQRY

SEQ ID NO: 14 - protein, 27 aa, Homo sapiens
DDASPDELNRYYASLRHYLNLVTRQRY

SEQ ID NO: 15 - protein, 31 aa, Homo sapiens
DAPGEDATPEELNRYYASLRHYLNLVTRQRY

SEQ ID NO: 16 - protein, 33 aa, Homo sapiens
NPEAPGEDASPDELNRYYASLRHYLNLVTRQRY

SEQ ID NO: 17 - protein, 34 aa, Homo sapiens
LKPEAPGDDASPEELNRYYASLRHYLNLVTRQRY

SEQ ID NO: 18 - protein, 15 aa, Artificial Sequence
  Description of Artificial Sequence Synthetic peptide
ASLRHWLNLVTRQRY

SEQ ID NO: 19 - protein, 15 aa, Artificial Sequence
  Description of Artificial Sequence Synthetic peptide
ASLRHXLNLVTRQRY

SEQ ID NO: 20 - protein, 15 aa, Artificial Sequence
  Description of Artificial Sequence Synthetic peptide
YSLRHFLNLVTRQRY

SEQ ID NO: 21 - protein, 15 aa, Artificial Sequence
  Description of Artificial Sequence Synthetic peptide
FSLRHFLNLVTRQRY

SEQ ID NO: 22 - protein, 12 aa, Artificial Sequence
  Description of Artificial Sequence Synthetic peptide
RHFLNLVTRQRY

SEQ ID NO: 23 - protein, 15 aa, Artificial Sequence
  Description of Artificial Sequence Synthetic peptide
ASLRHWLNLVTRQRY

SEQ ID NO: 24 - protein, 36 aa, Artificial Sequence
  Description of Artificial Sequence Synthetic peptide
YPAKPEAPGEDASPEELSRYYASLRHYLNLVTRQRY

SEQ ID NO: 25 - protein, 15 aa, Artificial Sequence
  Description of Artificial Sequence Synthetic peptide
ASLRHYLNLVARQRY

SEQ ID NO: 26 - protein, 15 aa, Artificial Sequence
  Description of Artificial Sequence Synthetic peptide
AALRHYLNLVARQRY

SEQ ID NO: 27 - protein, 15 aa, Artificial Sequence
  Description of Artificial Sequence Synthetic peptide
ASLRHYENLVTRQRY

SEQ ID NO: 28 - protein, 15 aa, Artificial Sequence
  Description of Artificial Sequence Synthetic peptide
ASLRHYLNLVTRQRY

SEQ ID NO: 29 - protein, 14 aa, Artificial Sequence
  Description of Artificial Sequence Synthetic peptide
ASLRFYLNLVTRQR

SEQ ID NO: 30 - protein, 15 aa, Artificial Sequence
  Description of Artificial Sequence Synthetic peptide
ASLRHYENLVTRQRY

SEQ ID NO: 31 - protein, 15 aa, Artificial Sequence
  Description of Artificial Sequence Synthetic peptide
ASLRHFENLVTRQRY

SEQ ID NO: 32 - protein, 15 aa, Artificial Sequence
  Description of Artificial Sequence Synthetic peptide
ASLRHYENLVTRQRY

SEQ ID NO: 33 - protein, 15 aa, Artificial Sequence
  Description of Artificial Sequence Synthetic peptide
ASLRHYLNLVTRQRY

SEQ ID NO: 34 - protein, 14 aa, Artificial Sequence
  Description of Artificial Sequence Synthetic peptide
ASLRHYLNLVTRQR

SEQ ID NO: 35 - protein, 15 aa, Artificial Sequence
  Description of Artificial Sequence Synthetic peptide
ASLRHFENLVTRQRY

SEQ ID NO: 36 - protein, 15 aa, Artificial Sequence
  Description of Artificial Sequence Synthetic peptide
ASLRHYLNLVTRQRY

SEQ ID NO: 37 - protein, 15 aa, Artificial Sequence
  Description of Artificial Sequence Synthetic peptide
ASLRHXLNLVTRQRY

SEQ ID NO: 38 - protein, 15 aa, Artificial Sequence
  Description of Artificial Sequence Synthetic peptide
ASLRHXLNLVTRQRY

SEQ ID NO: 39 - protein, 15 aa, Artificial Sequence
  Description of Artificial Sequence Synthetic peptide
ASLRHXLNLVTRQRY

SEQ ID NO: 40 - protein, 15 aa, Artificial Sequence
  Description of Artificial Sequence Synthetic peptide
ASLRHWLNLVTRQRY

SEQ ID NO: 41 - protein, 15 aa, Artificial Sequence
  Description of Artificial Sequence Synthetic peptide
ASLRNXLNLVTRQRY

SEQ ID NO: 42 - protein, 15 aa, Artificial Sequence
  Description of Artificial Sequence Synthetic peptide
ASLRHXLNLVTRQRY

SEQ ID NO: 43 - protein, 11 aa, Artificial Sequence
  Description of Artificial Sequence Synthetic peptide
HFLNLVTRQRY

SEQ ID NO: 44 - protein, 15 aa, Artificial Sequence
  Description of Artificial Sequence Synthetic peptide
ASLRHFLNLVTRQRX

SEQ ID NO: 45 - protein, 15 aa, Artificial Sequence
  Description of Artificial Sequence Synthetic peptide
ASLRXFLNLVTRQRY

SEQ ID NO: 46 - protein, 15 aa, Artificial Sequence
  Description of Artificial Sequence Synthetic peptide
ASLRHXLNLVTRQRY

SEQ ID NO: 47 - protein, 15 aa, Artificial Sequence
  Description of Artificial Sequence Synthetic peptide
FSLRNFLNLVTRQRY

SEQ ID NO: 48 - protein, 15 aa, Artificial Sequence
  Description of Artificial Sequence Synthetic peptide
YSLRHFLNLVTRQRY

SEQ ID NO: 49 - protein, 15 aa, Artificial Sequence
  Description of Artificial Sequence Synthetic peptide
ASLRHYWNLVTRQRY

SEQ ID NO: 50 - protein, 15 aa, Artificial Sequence
  Description of Artificial Sequence Synthetic peptide
ASLRHYLNWVTRQRY

SEQ ID NO: 51 - protein, 15 aa, Artificial Sequence
  Description of Artificial Sequence Synthetic peptide
ASLRAFLNLVTRQRY

SEQ ID NO: 52 - protein, 15 aa, Artificial Sequence
  Description of Artificial Sequence Synthetic peptide
ASLRHXLNLVTRQRY

SEQ ID NO: 53 - protein, 15 aa, Artificial Sequence
  Description of Artificial Sequence Synthetic peptide
ASLRHFLNLVTRQRY

SEQ ID NO: 54 - protein, 15 aa, Artificial Sequence
  Description of Artificial Sequence Synthetic peptide
ASLRHFLNLVTRQRF

SEQ ID NO: 55 - protein, 15 aa, Artificial Sequence
  Description of Artificial Sequence Synthetic peptide
ASLRHFLNLVWRQRY

SEQ ID NO: 56 - protein, 36 aa, Rattus sp.; identical to SEQ ID NO: 24

SEQ ID NO: 57 - protein, 36 aa, Sus sp.; identical to SEQ ID NO: 24

SEQ ID NO: 58 - protein, 36 aa, Cavia sp.
YPSKPEAPGSDASPEELARYYASLRHYLNLVTRQRY

SEQ ID NO: 59 - protein, 36 aa, Homo sapiens
YPSKPDNPGEDAPAEDMARYYSALRHYINLITRQRY

SEQ ID NO: 60 - protein, 36 aa, Rattus sp.; identical to SEQ ID NO: 59

SEQ ID NO: 61 - protein, 36 aa, Oryctolagus sp.; identical to SEQ ID NO: 59

SEQ ID NO: 62 - protein, 36 aa, Canis sp.; identical to SEQ ID NO: 59

SEQ ID NO: 63 - protein, 36 aa, Sus sp.
YPSKPDNPGEDAPAEDLARYYSALRHYINLITRQRY

SEQ ID NO: 64 - protein, 36 aa, Bos sp.; identical to SEQ ID NO: 63

SEQ ID NO: 65 - protein, 36 aa, Ovis sp.
YPSKPDNPGDDAPAEDLARYYSALRHYINLITRQRY

SEQ ID NO: 66 - protein, 36 aa, Cavia sp.; identical to SEQ ID NO: 59

SEQ ID NO: 67 - protein, 36 aa, Homo sapiens
ASLEPEYPGDNATPEQMAQYAAELRRYINMLTRPRY

SEQ ID NO: 68 - protein, 36 aa, Ovis sp.
APLEPVYPGDNATPEQMAQYAADLRRYINMLTRPRY

SEQ ID NO: 69 - protein, 35 aa, Sus sp.
APLEPVYPGDDATPEMAQYAAELRRYINMLTRPRY

SEQ ID NO: 70 - protein, 36 aa, Canis sp.
APLEPVYPGDDATPEQMAQYAAELRRYINMLTRPRY

SEQ ID NO: 71 - protein, 36 aa, Felis sp.
APLEPVYPGDNATPEQMAQYAAELRRYINMLTRPRY

SEQ ID NO: 72 - protein, 36 aa, Bos sp.
APLEPEYPGDDATPEQMAQYAAELRRYINMLTRPRY

SEQ ID NO: 73 - protein, 36 aa, Rattus sp.
APLEPMYPGDYATHEQRAQYETQLRRYINTLTRPRY

SEQ ID NO: 74 - protein, 36 aa, Mus sp.; identical to SEQ ID NO: 73

SEQ ID NO: 75 - protein, 36 aa, Cavia sp.
APLEPMYPGDYATPEQMAQYETQLRRYINTLTRPRY

SEQ ID NO: 76 - protein, 4 aa, Artificial Sequence
  Description of Artificial Sequence Synthetic peptide
AAPV

(19) United States
(12) Patent Application Publication
(10) Pub. No.: US 2009/0054326 A1
(43) Pub. Date: Feb. 26, 2009

(54) FORMULATIONS FOR ENHANCED MUCOSAL DELIVERY OF PYY

(75) Inventors: Henry R. Costantino, Woodinville, WA (US); Mary S. Kleppe, Oaksdale, CT (US); Annemarie S. Cohen, Kirkland, WA (US); Anthony P. Sileno, Brookhaven Hamlet, NY (US)

Correspondence Address: NASTECH PHARMACEUTICAL COMPANY INC;MDRNA, Inc., 3830 MONTE VILLA PARKWAY, BOTHELL, WA, 98021-7266, US

(73) Assignee: NASTECH PHARMACEUTICAL COMPANY INC., Bothell, WA (US)

(21) Appl. No.: 11/995,315

(22) PCT Filed: Jul. 10, 2006
(86) PCT No.: PCT/US06/26707

§ 371 Date: Sep. 16, 2008

Related U.S. Application Data
(60) Provisional application No. 60/698,052, filed on Jul. 11, 2005.
(60) Provisional application No. 60/699,294, filed on Jul. 13, 2005.

(51) Int. Cl. A61K 38/00; A61P 3/00

(52) U.S. Cl. 514/12

(57) ABSTRACT

Pharmaceutical formulations are described for enhancing mucosal delivery of peptide YY (PYY) to a mammal. A PYY dosage form is described that is suitable for multi-use administration. The PYY dosage form comprises a bottle containing an aqueous pharmaceutical formulation and an actuator effective intranasal administration of the formulation. The formulation comprises a therapeutically effective amount of PYY, a buffer to control pH, a water-miscible polar organic solvent and a chelating agent for cations. The PYY dosage form exhibits at least 90% PYY recovery after storage as used for greater than about five days.

### BACKGROUND OF THE INVENTION

Obesity and its associated disorders are common and very serious public health problems in the United States and throughout the world. It has been shown that certain peptides that bind to the Y2 receptor when administered peripherally to a mammal induce weight loss. The Y2 receptor-binding peptides are neuropeptides that bind to the Y2 receptor. These Y2 receptor-binding peptides belong to a family of peptides including peptide YY (PYY), neuropeptide Y (NPY), and pancreatic peptide (PP).

These approximately 36 amino acid peptides have a compact helical structure involving a “PP-fold” in the middle of the peptide. Specific features include a polyproline helix in residues 1 through 8, a β-turn in residues 9 through 14, an α-helix in residues 15 through 30, an outward-projecting C-terminus in residues 30 through 36, and a carboxyl terminal amide, which appears to be critical for biological activity. It has been shown that a 36 amino acid peptide called Peptide YY(1-36) [PYY(1-36)] [YPIKPEAPGEDASPEELNRYYASLRHYLNLVTRQRY, (SEQ ID NO: 1)] when administered peripherally by injection to an individual produces weight loss and thus can be used as a drug to treat obesity and related diseases, Morley, J., Neuropsychobiology 21:22-30 (1989). It was later found that to produce this effect PYY bound to a Y2 receptor, and the binding of a Y2 agonist to the Y2 receptor caused a decrease in the ingestion of carbohydrate, protein and meal size, Leibowitz, S. F. et al., Peptides 12:1251-1260 (1991). An alternate molecular form of PYY is PYY(3-36) IKPEAPGEDASPEELNRYYASLRHYLNLVTRQRY (residues 3-36 of SEQ ID NO: 1), Eberlein, Eysselein et al., Peptides 10:797-803, 1989). Hereinafter the term PYY refers to full-length PYY and any fragment of PYY that binds to a Y2 receptor.

It is known that PYY can be administered by intravenous infusion or injection to treat life-threatening hypotension as encountered in shock, especially that caused by endotoxins (U.S. Pat. No. 4,839,343), to inhibit proliferation of pancreatic tumors in mammals by perfusion, parenteral, intravenous, or subcutaneous administration, and by implantation (U.S. Pat. No. 5,574,010) and to treat obesity (Morley (1989)) and U.S. Patent Application No. 20020141985). It is also claimed that PYY can be administered by parenteral, oral, nasal, rectal and topical routes to domesticated animals or humans in an amount effective to increase weight gain of said subject by enhancing gastrointestinal absorption of a sodium-dependent cotransported nutrient (U.S. Pat. No. 5,912,227). However, for the treatment of obesity and related diseases, including diabetes, the mode of administration has been limited to intravenous IV infusion with no effective formulations optimized for alternative administration of PYY. None of these prior art teachings provide formulations that contain PYY or PYY(3-36) combined with excipients designed to enhance mucosal (i.e., nasal, buccal, oral) delivery nor do they teach the value of endotoxin-free Y2-receptor binding peptide formulations for non-infused administration.

Previously, formulations for intra-nasal administration of PYY were described in patent applications, including Patent Application Publication Nos. WO040563142; US2004/0115135; US2004/0157777; US2004/0209807; and US2005/0002927, herein incorporated by reference. These applications disclose formulations suitable for dosing between 20 and 200 μg in 0.1 ml, i.e., with concentrations between 0.2 and 2.0 mg/ml PYY. The stability of the dosage form of 0.3 mg/ml PYY was tested at various pH values over five days at 40° C. in a formulation comprising 10 mM citrate and 100 mM NaCl. The pH optimum was found at 4.9 wherein greater than 80% of the peptide remained following the five day incubation. However, it was found subsequently that PYY stability was substantially influenced by PYY concentration: at higher concentration, PYY stability decreased. Moreover, the formulations previously described and tested for intranasal administration included excipients such as methyl-β-cyclodextrin and L-α-phosphatidylcholine didecanoyl, which are not generally regarded as safe (GRAS) excipients. Accordingly, to provide dosage forms and formulations that have general utility under common conditions for a pharmaceutical drug, a compelling need arose to develop alternate formulation compositions having improved stability. Further, a compelling need arose to develop formulations and dosage forms comprising GRAS excipients as a distinct alternative to using non-compendial excipients.

### BRIEF DESCRIPTION OF THE DRAWINGS

FIG. 1: PYY3-36 permeation of formulations tested in Example 1.

FIG. 2: PYY3-36 permeation of formulations tested in Example 2.

FIG. 3: PYY3-36 permeation of formulations tested in Example 3.

FIG. 4: PYY3-36 permeation of formulations tested in Example 4.

FIG. 5. Peptide recovery vs. time: (A) 25° C. storage; (B) 40° C. storage; and (C) 50° C. storage.

FIG. 6. Peptide recovery vs. time, 40° C. storage: (A) citrate buffer-based formulations; (B) acetate buffer-based formulations; (C) glutamate buffer-based formulations; and (D) unbuffered formulations.

FIG. 7. PYY3-36 stability at elevated temperature and atomization stress of thrice daily spraying for samples tested in Example 8.

FIG. 8. PYY3-36 stability at elevated temperature and atomization stress of thrice daily spraying for samples tested in Example 9.

FIG. 9. PYY3-36 stability at elevated temperature and atomization stress of thrice daily spraying, tested in Example 10: (A) samples 5-1, 5-2 and 5-3; (B) samples 5-4, 5-5, 5-6 and 5-7; (C) samples 5-8, 5-9, 5-10 and 5-11; (D) samples 5-12, 5-13, and 5-14.

### DETAILED DESCRIPTION OF THE INVENTION

In order to provide better understanding of the present invention, the following definitions and detailed description are provided:

### Y2 Receptor-Binding Peptides

The Y2 receptor-binding peptides used in mucosal formulations of the present invention include three naturally occurring bioactive peptide families, PP, NPY, and PYY. Examples of Y2 receptor-binding peptides and their uses are described in U.S. Pat. No. 5,026,685; U.S. Pat. No. 5,574,010; U.S. Pat. No. 5,604,203; U.S. Pat. No. 5,696,093; U.S. Pat. No. 6,046,167; Gehlert et. al., Proc. Soc. Exp. Biol. Med. 218:7-22 (1998); Sheikh et al., Am. J. Physiol. 261:701-15 (1991); Fournier et al., Mol. Pharmacol. 45:93-101 (1994); Kirby et al., J. Med. Chem. 38:4579-4586 (1995); Rist et al., Eur. J. Biochem. 247:1019-1028 (1997); Kirby et al., J. Med. Chem. 36:3802-3808 (1993); Grundemar et al., Regulatory Peptides 62:131-136 (1996); U.S. Pat. No. 5,696,093 (examples of PYY agonists), U.S. Pat. No. 6,046,167. According to the present invention a Y2 receptor-binding peptide includes the free bases, acid addition salts or metal salts, such as potassium or sodium salts or the peptides Y2 receptor-binding peptides that have been modified by such processes as amidation, glycosylation, acylation, sulfation, phosphorylation, acetylation and cyclization, (U.S. Pat. No. 6,093,692; and U.S. Pat. No. 6,225,445 and pegylation).

### Peptide YY Agonists

As used herein, “PYY” refers to PYY(1-36) (SEQ ID NO: 1) in native-sequence or in variant form, as well as derivatives, fragments, and analogs of PYY from any source, whether natural, synthetic, or recombinant. The PYY is comprised of at least the last 15 amino acid residues or analogs thereof of the PYY sequence, PYY(22-36). Other PYY peptides, which may be used are PYY(1-36) (SEQ ID NO: 1), PYY(3-36), PYY(4-36), PYY(5-36), PYY(6-36), PYY(7-36), PYY(8-36), PYY(9-36), PYY(10-36), PYY(11-36), PYY(12-36), PYY(13-36), PYY(14-36), PYY(15-36), PYY(16-36), PYY(17-36), PYY(18-36), PYY(19-36), PYY(20-36), and PYY(21-36). These peptides typically bind to the Y receptors in the brain and elsewhere, especially the Y2 and/or Y5 receptors. Typically these peptides are synthesized in endotoxin-free or pyrogen-free forms although this is not always necessary.

Other PYY peptides include those PYY peptides in which conservative amino acid residue changes have been made, for example, site specific mutation of a PYY peptide including [Asp¹⁵] PYY(15-36) (SEQ ID NO: 2), [Thr¹³] PYY(13-36) (SEQ ID NO: 3), [Val¹²] PYY(12-36) (SEQ ID NO: 4), [Glu¹¹] PYY(11-36) (SEQ ID NO: 5), [Asp¹⁰] PYY(10-36) (SEQ ID NO: 6), [Val⁷] PYY(7-36) (SEQ ID NO: 7), [Asp⁶] PYY(6-36) (SEQ ID NO: 8), [Gln⁴] PYY(4-36) (SEQ ID NO: 9), [Arg⁴] PYY(4-36) (SEQ ID NO: 10), [Asn⁴] PYY(4-36) (SEQ ID NO: 11), [Val³] PYY(3-36) (SEQ ID NO: 12) and [Leu³] PYY(3-36) (SEQ ID NO: 13). Other PYY peptides include those peptides in which at least two conservative amino acid residue changes have been made including [Asp¹⁰, Asp¹⁵] PYY(10-36) (SEQ ID NO: 14), [Asp⁶, Thr¹³] PYY(6-36) (SEQ ID NO: 15), [Asn⁴, Asp¹⁵] PYY(4-36) (SEQ ID NO: 16), and [Leu³, Asp¹⁰] PYY(3-36) (SEQ ID NO: 17).

Also included are analogs of a PYY for example those disclosed in U.S. Pat. Nos. 5,604,203 and 5,574,010. These include the following peptides:

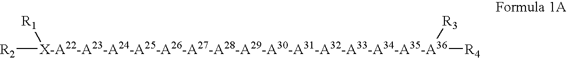

For Formula 1A the following PYY(22-36) peptide analogs can be created where:

X is Cys or is deleted; each of R₁ and R₂ is bonded to the nitrogen atom of the alpha-amino group of the N-terminal amino acid; R₁ is H, C₁-C₁₂ alkyl, C₆-C₁₈ aryl, C₁-C₁₂ acyl, C₇-C₁₈ aralkyl, or C₇-C₁₈ alkaryl; R₂ is H, C₁-C₁₂ alkyl, C₆-C₁₈ aryl, C₁-C₁₂ acyl, C₇-C₈ aralkyl, or C₇-C₁₈ alkaryl; A²² is an aromatic amino acid, Ala, Aib, Anb, N-Me-Ala, or is deleted; A²³ is Ser, Thr, Ala, Aib, N-Me-Ser, N-Me-Thr, N-Me-Ala, D-Trp, or is deleted; A²⁴ is Leu, Gly, Ile, Val, Trp, Nle, Nva, Aib, Anb, N-Me-Leu, or is deleted; A²⁵ is Arg, Lys, homo-Arg, diethyl-homo-Arg, Lys-epsilon-NH—R (where R is H, a branched or straight chain C₁-C₁₀ alkyl group, or an aryl group), Orn, or is deleted; A²⁶ is Ala, His, Thr, 3-Me-His, 1-Me-His, beta-pyrozolylalanine, N-Me-His, Arg, Lys, homo-Arg, diethyl-homo-Arg, Lys-epsilon-NH—R (where R is H, a branched or straight chain C₁-C₁₀ alkyl group, or an aryl group), Orn, or is deleted; A²⁷ is NaI, Bip, Pcp, Tic, Trp, Bth, Thi, or Dip; A²⁸ is Leu, Val, Trp, Nle, Nva, Aib, Anb, or N-Me-Leu; A²⁹ is Asn, Ala, Gln, Gly, Trp, or N-Me-Asn; A³⁰ is Leu, Ile, Val, Trp, Nle, Nva, Aib, Anb, or N-Me-Leu; A³¹ is Val, Leu, Ile, Trp, Nle, Nva, Aib, Anb, or N-Me-Val; A³² is Thr, Ser, N-Me-Ser, N-Me-Thr, or D-Trp; A³³ is Cys, Arg, Lys, homo-Arg, diethyl-homo-Arg, Lys-epsilon-NH—R (where R is H, a branched or straight chain C₁-C₁₀ alkyl group, or C₆-C₁₈ aryl group), or Orn; A³⁴ is Cys, Gln, Asn, Ala, Gly, N-Me-Gin, Aib, or Anb; A³⁵ is Cys, Arg, Lys, homo-Arg, diethyl-homo-Arg, Lys-epsilon-NH—R (where R is H, a branched or straight chain C₁-C₁₀ alkyl group, or C₆-C₁₈ aryl group), or Orn; A³⁶ is an aromatic amino acid, or Cys; R₃ is H, C₁-C₁₂ alkyl, C₆-C₁₈ aryl, C₁-C₁₂ acyl, C₇-C₁₈ aralkyl, or C₇-C₁₈ alkaryl; R is H, C₁-C₁₂ alkyl, C₆-C₁₈ aryl, C₁-C₁₂ acyl, C₇-C₁₈ aralkyl, or C₇-C₁₈ alkaryl, or a pharmaceutically acceptable salt thereof.

Examples of PYY(22-36) Formula 1A peptide analogs include

N-alpha-Ac-Ala-Ser-Leu-Arg-His-Trp-Leu-Asn-Leu-Val-Thr-Arg-Gln-Arg-Tyr-NH₂ (SEQ ID NO: 18); N-alpha-Ac-Ala-Ser-Leu-Arg-His-Thi-Leu-Asn-Leu-Val-Thr-Arg-Gln-Arg-Tyr-NH₂ (SEQ ID NO: 19), or pharmaceutically acceptable salts thereof.

Additional Formula 1A analogs may be used including:

Where the —CO—NH— bond between the residues A²⁸ and A²⁹, A²⁹ and A³⁰, A³⁰ and A³¹, A³¹ and A³², A³³ and A³⁴, A³⁴ and A³⁵, or A³⁵ and A³⁶ is replaced with CH₂—NH, CH₂—S, CH₂—CH₂, or CH₂—O, or where the CO—NH bond between the residues A³⁵ and A³⁶ is replaced with CH₂—NH.

For Formula 1B, the following PYY(22-36) peptide analogs can be created where:

X is Cys or is deleted; R₁ and R₂ are bonded to the N-terminal amino acid; R₁ is H, C₁-C₁₂ alkyl, C₆-C₁₈ aryl, C₁-C₁₂ acyl, C₇-C₁₈ aralkyl, or C₇-C₁₈ alkaryl; R₂ is H, C₁-C₁₂ alkyl, C₆-C₁₈ aryl, C₁-C₁₂ acyl, C₇-C₁₈ aralkyl, or C₇-C₁₈ alkaryl; A²² is an aromatic amino acid or is deleted; A²³ is Ser, Thr, Ala, Aib, N-Me-Ser, N-Me-Thr, Me-Ala, D-Tip, or is deleted; A²⁴ is Leu, Gly, Ile, Val, Trp, Nle, Nva, Aib, Anb, N-Me-Leu, or is deleted; A²⁵ is Arg, Lys, homo-Arg, diethyl-homo-Arg, Lys-epsilon-NH—R (where R is H, a branched or straight chain C₁-C₁₀ alkyl group, or an aryl group), Orn, or is deleted; A²⁶ is Ala, His, Thr, 3-Me-His, 1-Me-His, beta-pyrozolylalanine, N-Me-His, Arg, Lys, homo-Arg, diethyl-homo-Arg, Lys-epsilon-NH—R (where R is H, a branched or straight chain C₁-C₁₀ alkyl group, or an aryl group), or Orn; A²⁷ is an aromatic amino acid other than Tyr; A²⁸ is Leu, Val, Trp, Nle, Nva, Aib, Anb, or N-Me-Leu; A²⁹ is Asn, Ala, Gln, Gly, Trp, or N-Me-Asn; A³⁰ is Leu, Ile, Val, Trp, Nle, Nva, Aib, Anb, or N-Me-Leu; A³¹ is Val, Leu, Ile, Trp, Nle, Nva, Aib, Anb, or N-Me-Val; A³² is Thr, Ser, N-Me-Ser, N-Me-Thr, or D-Trp; A³³ is Cys, Arg, Lys, homo-Arg, diethyl-homo-Arg, Lys-epsilon-NH—R (where R is H, a branched or straight chain C₁-C₁₀ alkyl group, or C₆-C₁₈ aryl group), or Orn; A³⁴ is Cys, Gln, Asn, Ala, Gly, N-Me-Gin, Aib, or Anb; A³⁵ is Cys, Arg, Lys, homo-Arg, diethyl-homo-Arg, Lys-epsilon-NH—R (where R is H, a branched or straight chain C₁-C₁₀ alkyl group, or C₆-C₁₈ aryl group), or Orn; A³⁶ is an aromatic amino acid, or Cys; R₃ is H, C₁-C₁₂ alkyl, C₆-C₁₈ aryl, C₁-C₁₂ acyl, C₇-C₁₈ aralkyl, or C₇-C₁₈ alkaryl; R₄ is H, C₁-C₁₂ alkyl, C₆-C₁₈ aryl, C₁-C₁₂ acyl, C₇-C₁₈ aralkyl, or C₇-C₁₈ alkaryl, or a pharmaceutically acceptable salt thereof.

Examples of a PYY(22-36) Formula 1B peptide analog includes:

N-alpha-Ac-Tyr-Ser-Leu-Arg-His-Phe-Leu-Asn-Leu-Val-Thr-Arg-Gln-Arg-Tyr-NH₂ (SEQ ID NO: 20), or a pharmaceutically acceptable salt thereof.

Additional Formula 1B analogs can be used, wherein A²⁷ is Phe, NaI, Bip, Pcp, Tic, Trp, Bth, Thi, or Dip.

Another example of a PYY(22-36) Formula 1B peptide analog includes:

(SEQ ID NO: 21)

N-alpha-Ac-Phe-Ser-Leu-Arg-His-Phe-Leu-Asn-Leu-
Val-Thr-Arg-Gln-Arg-Tyr-NH₂.

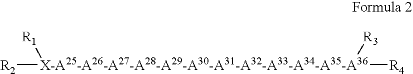

For Formula 2 the following PYY(25-36) peptide analogs can be created where:

R₁ and R₂ is bonded to the nitrogen atom of the alpha-amino group of the N-terminal amino acid; R₁ is H, C₁-C₁₂ alkyl, C₆-C₁₈ aryl, C₁-C₁₂ acyl, C₇-C₁₈ aralkyl, or C₇-C₁₈ alkaryl; R₂ is H, C₁-C₁₂ alkyl, C₆-C₁₈ aryl, C₁-C₁₂ acyl, C₇-C₁₈ aralkyl, or C₇-C₁₈ alkaryl; A²⁵ is Arg, Lys, homo-Arg, diethyl-homo-Arg, Lys-epsilon-NH—R (where R is H, a branched or straight chain C₁-C₁₀ alkyl group, or an aryl group), Orn, or is deleted; A²⁶ is Ala, His, Thr, 3-Me-His, beta-pyrozolylalanine, N-Me-His, Arg, Lys, homo-Arg, diethyl-homo-Arg, Lys-epsilon-NH—R (where R is H, a branched or straight chain C₁-C₁₀ alkyl group, or an aryl group), or Orn; A²⁷ is an aromatic amino acid; A²⁸ is Leu, Val, Trp, Nle, Nva, Aib, Aib, Anb, or N-Me-Leu; A²⁹ is Asn, Ala, Gln, Gly, Trp, or N-Me-Asn; A³⁰ is Leu, Ile, Val, Trp, Nle, Nva, Aib, Anb, or N-Me-Leu; A³¹ is Val, Leu, Ile, Trp, Nle, Nva, Aib, Anb, or N-Me-Val; A³² is Thr, Ser, N-Me-Ser, N-Me-Thr, or D-Trp; A³³ is Arg, Lys, homo-Arg, diethyl-homo-Arg, Lys-epsilon-NH—R (where R is H, a branched or straight chain C₁-C₁₀ alkyl group, or C₆-C₁₈ aryl group), Cys, or Orn; A³⁴ is Cys, Gln, Asn, Ala, Gly, N-Me-Gin, Aib, or Anb; A³⁵ is Arg, Lys, homo-Arg, diethyl-homo-Arg, Lys-epsilon-NH—R (where R is H, a branched or straight chain C₁-C₁₀ alkyl group, or C₆-C₁₈ aryl group), Cys, or Orn; A³⁶ is an aromatic amino acid, or Cys; R₃ is H, C₁-C₁₂ alkyl, C₆-C₁₈ aryl, C₁-C₁₂ acyl, C₇-C₁₈ aralkyl, or C₇-C₁₈ alkaryl; and R₄ is H, C₁-C₁₂ alkyl, C₆-C₁₈ aryl, C₁-C₁₂ acyl, C₇-C₁₈ aralkyl, or C₇-C₁₈ alkaryl, or a pharmaceutically acceptable salt thereof.

Additional analogs can be used wherein A²⁷ of Formula 2 is Phe, NaI, Bip, Pcp, Tic, Trp, Bth, Thi, or Dip.

An examples of a PYY(22-36) Formula 2 analog includes:

N-alpha-Ac-Arg-His-Phe-Leu-Asn-Leu-Val-Thr-Arg-Gln-Arg-TYr-NH₂ (SEQ. ID. NO: 22), or a pharmaceutically acceptable salt thereof.

Additional Formula 2 analogs may be used including:

Where the —CO—NH— bond between the residues A²⁸ and A²⁹, A²⁹ and A³⁰, A³⁰ and A³¹, A³¹ and A³², A³² and A³³, A³³ and A³⁴, A³⁴ and A³⁵, or A³⁵ and A³⁶ is replaced with CH₂—NH, CH₂—S, CH₂—CH₂, or CH₂—O.

Further, analogs may include dimeric compounds comprising either two peptides of Formula 1A, Formula 1B, or Formula 2, or one peptide of Formula 1A and one peptide of Formula 1B, or one peptide of Formula 1A and one peptide of Formula 2, or one peptide of Formula 1B and one peptide of Formula 2; wherein said dimer is formed by either an amide bond or a disulfide bridge between said two peptides.

Abbreviations included Asp=D=Aspartic Acid; Ala=A=Alanine; Arg=R=Arginine; Asn=N=Asparagine; Cys=C=Cysteine; Gly=G=Glycine; Glu=E=Glutamic Acid; Gln=Q=Glutamine; His=H=Histidine; Ile=I=soleucine; Leu=L=Leucine; Lys=K=Lysine; Met=M=Methionine; Phe=F=Phenylalanine; Pro=P=Proline; Ser=S=Serine; Thr=T=Tbreonine; Trp=W=Tryptophan; Tyr=Y=Tyrosine; Val=V=Valine; Orn=Ornithine; NaI=2-napthylalanine; Nva=Norvaline; Nle=Norleucine; Thi=2-thienylalanine; Pcp=4-chlorophenylalanine; Bth=3-benzothienyalanine; Bip=4,4′-biphenylalanine; Tic=tetrahydroisoquinoline-3-carboxylic acid; Aib=aminoisobutyric acid; Anb=.alpha.-aminonormalbutyric acid; Dip=2,2-diphenylalanine; and Thz=4-Thiazolylalanine (U.S. Pat. No. 5,604,203).

Analogs described in U.S. Pat. No. 5,574,010 include the following:

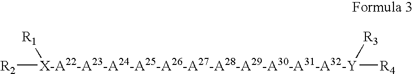

Analogs of Formula 3 wherein X is a chain of 0-5 amino acids, inclusive, the N-terminal one of which is bonded to R₁ and R₂; Y is a chain of 0-4 amino acids, inclusive, the C-terminal one of which is bonded to R₃ and R₄; R₁ is H, C₁-C₂ alkyl (e.g., methyl), C₆-C₁₈ aryl (e.g., phenyl, napthaleneacetyl), C₁-C₁₂ acyl (e.g., formyl, acetyl, and myristoyl), C₇-C₁₈ aralkyl (e.g., benzyl), or C₇-C₁₈ alkaryl (e.g., p-methylphenyl); R₂ is H, C₁-C₁₂ alkyl (e.g., methyl), C₆-C₁₈ aryl (e.g., phenyl, naphthaleneacetyl), C₁-C₁₂ acyl (e.g., formyl, acetyl, and myristoyl), C₇-C₁₈ aralkyl (e.g., benzyl), or C₇-C₁₈ alkaryl (e.g., p-methylphenyl); A²² is an aromatic amino acid, Ala, Aib, Anb, N-Me-Ala, or is deleted; A²³ is Ser, Thr, Ala, N-Me-Ser, N-Me-Thr, N-Me-Ala, or is deleted; A²⁴ is Leu, lie, Vat, Trp, Gly, Aib, Anb, N-Me-Leu, or is deleted; A²⁵ is Arg, Lys, homo-Arg, diethyl-homo-Arg, Lys-epsilon-NH—R (where R is H, a branched or straight chain C₁-C₁₀ alkyl group, or an aryl group), Orn, or is deleted; A²⁶ is His, Thr, 3-Me-His, 1-Me-His, beta-pyrozolylalanine, N-Me-His, Arg, Lys, homo-Arg, diethyl-homo-Arg, Lys-epsilon-NH—R (where R is H, a branched or straight chain C₁-C₁₀ alkyl group, or an aryl group), Orn, or is deleted; A²⁷ is an aromatic amino acid other than Tyr; A²⁸ is Leu, Ile, Vat, Trp, Aib, Aib, Anb, or N-Me-Leu; A²⁹ is Asn, Ala, Gln, Gly, Trp, or N-Me-Asn; A³⁰ is Leu, Ile, Val, Trp, Aib, Anb, or N-Me-Leu; A³¹ is Vat, Ile, Trp, Aib, Anb, or N-Me-Val; A³² is Thr, Ser, N-Me-Set, or N-Me-Thr; R₃ is H, C₁-C₁₂ alkyl (e.g., methyl), C₆-C₁₈ aryl (e.g., phenyl, naphthaleneacetyl), C₁-C₁₂ acyl (e.g., formyl, acetyl, and myristoyl), C₇-C₁₈ aralkyl (e.g., benzyl), or C₇-C₁₈ alkaryl (e.g., p-methylphenyl); R₄ is H, C₁-C₁₂ alkyl (e.g., methyl), C₆-C₁₈ aryl (e.g., phenyl, naphthaleneacetyl), C₁-C₁₂ acyl (e.g., formyl, acetyl, and myristoyl), C₇-C₁₈ aralkyl (e.g., benzyl), or C₇-C₁₈ alkaryl (e.g., p-methylphenyl), or a pharmaceutically acceptable salt thereof.

Particularly preferred analogs of Formula 3 include:

(SEQ. ID. NO: 23)

N-.alpha.-Ala-Ser-Leu-Arg-His-Trp-Leu-Asn-Leu-Val-
Thr-Arg-Gln-Arg-Tyr-NH₂.

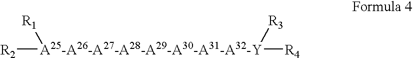

Another peptide YY analog is Formula 4 where the N-terminal amino acid bonds to R₁ and R₂; Y is a chain of 0-4 amino acids, inclusive the C-terminal one of which bonds to R₃ and R₄; R₁ is H, C₁-C₁₂ alkyl, C₆-C₁₈ aryl, C₁-C₁₂ acyl, C₇-C₁₈ aralkyl, or C₇-C₁₈ alkaryl; R₂ is H, C₁-C₁₂ alkyl, C₆-C₁₈ aryl, C₁-C₁₂ acyl, C₇-C₁₈ aralkyl, or C₇-C₁₈ is alkaryl; A²⁵ is Arg, Lys, homo-Arg, diethyl-homo-Arg, Lys-epsilon-NH—R (where R is H, a branched or straight chain C₁-C₁₀ alkyl group, or an aryl group), Orn, or is deleted; A²⁶ is Ala, His, Thr, 3-Me-His, 1-Me-His, beta-pyrozolylalanine, N-Me-His, Arg, Lys, homo-Arg, diethyl-homo-Arg, Lys-epsilon-NH—R (where R is H, a branched or straight chain C₁-C₁₀ alkyl group, or an aryl group), Orn or is deleted; A²⁷ is an aromatic amino acid; A²⁸ is Leu, Ile, Val, Trp, Aib, Anb, or N-Me-Leu; A²⁹ is Asn, Ala, Gln, Gly, Trp, or N-Me-Asn; A³⁰ is Leu, Ile, Val, Trp, Aib, Anb, or N-Me-Leu; A³¹ is Val, Ile, Trp, Aib, Anb, or N-Me-Val; A³² is Thr, Set, N-Me-Set, or N-Me-Thr or D-Trp; R₃ is H, C₁-C₁₂ alkyl, C₆-C₁₈ aryl, C₁-C₁₂ acyl, C₇-C₁₈ aralkyl, or C₇-C₁₈ alkaryl; and R₄ is H, C₁-C₁₂ alkyl, C₆-C₁₈ aryl, C₁-C₁₂ acyl, C₇-C₁₈ aralkyl, or C₇-C₁₈ alkaryl, or a pharmaceutically acceptable salt thereof. Note that, unless indicated otherwise, for all peptide YY agonists described herein, each amino acid residue, e.g., Leu and A¹, represents the structure of NH—C(R)H—CO—, in which R is the side chain. Lines between amino acid residues represent peptide bonds which join the amino acids. Also, where the amino acid residue is optically active, it is the L-form configuration that is intended unless D-form is expressly designated.

Abbreviations: Aib=aminoisobutyric acid; Anb=.alpha.-aminonormalbutyric acid; Bip=4,4′-biphenylalanine; Bth=3-benzothienyalanine; Dip=2,2-diphenylalanine; Nat=2-napthylalanine; Orn=Ornithine; Pcp=4-chlorophenylalanine; Thi=2-thienylalanine; Tic=tetrahydroisoquinoline-3-carboxylic acid. (U.S. Pat. No. 5,574,010).

Examples of additional PYY synthesized analogs include: [im-DNP-His²⁶]PYY: YPAKPEAPGEDASPEELSRYYASLR [im-DNP-His²⁶]YLNLVMQRY-NH₂ (SEQ. ID No. 24); [Ala³²]PYY: A S L R H Y L N L V [Ala] R Q R Y-NH₂ (SEQ. ID No. 25); [Ala^{23,32}]PYY: A [Ala] L R H Y L N L V [Ala] R Q R Y-NN₂ (SEQ. ID No. 26); [Glu²⁸]PYY(22-36): A S L R H Y [Glu] N L V T R Q R Y-NH₂ (SEQ. ID No. 27); N-alpha-Ac-PYY(22-36): N-alpha-Ac-A S L R H Y L N L V T R Q R Y-NH₂ (SEQ. ID No. 28); N-alpha-Ac[p.CL.Phe.sup.26]PYY: N-alpha-Ac-A S L R [p.Cl.Phe²⁶] Y L N L V T R Q R (SEQ. ID No. 29); N-alpha-Ac[Glu²⁸]PYY: N-alpha-Ac-A S L R H Y [Glu] N L V T R Q R Y-NH₂ (SEQ. ID No. 30); N-alpha-Ac[Phe²⁷]PYY: N-alpha-Ac-A S L R H [Phe] E N L V T R Q R [N-Me-Tyr]-NH₂ (SEQ ID NO. 31ₙ); N-alpha-Ac]8N-Me-Tyr³⁶]PYY: N-alpha-Ac-A S L R H Y E N L V T R Q R [N-Me-Tyr]-NH₂ (SEQ. ID No. 32); N-alpha-myristoyl-PYY(22-36): N-alpha-myristoyl-A S L R H Y L N L V T R Q R Y-NH₂ (SEQ. ID No. 33); N-alpha-naphthateneacetyl-PYY(22-36): N-alpha-naphthateneacetyl-A S L R H Y L N L V T R Q R (SEQ. ID No. 34); N-alpha-Ac[Phe²⁷]PYY: N-alpha-Ac-A S L R H [Phe] E N L V T R Q R [N-Me-Tyr]-NH₂ (SEQ. ID No. 35); N-alpha-Ac-PYY(22-36): N-alpha-Ac-A S L R H Y L N L V T R Q R Y-NH₂ (SEQ. ID No. 36); N-alpha-Ac-[Bth²⁷]PYY(22-36): N-alpha-Ac-A S L R H [Bth] L N L V T R Q R Y-NH₂ (SEQ. ID No. 37); N-alpha-Ac-[Bip²⁷]PYY(22-36): N-alpha-Ac-A S L R H [Bip] L N L V T R Q R Y-NH₂ (SEQ. ID No. 38); N-alpha-Ac-[Nal²⁷]PYY(22-36): N-alpha-Ac-A S L R H [NaL] L N L V T R Q R Y-NH₂ (SEQ. ID No. 39); N-alpha-Ac-[Trp²⁷]PYY(22-36): N-alpha-Ac-A S L R H [Trp] L N L V T R Q R Y-NH₂ (SEQ. ID No. 40); N-alpha-Ac-[Thi²⁷]PYY(22-36): N-alpha-Ac-A S L R N [Thi] L N L V T R Q R Y-—NH₂ (SEQ. ID No. 41); N-alpha-Ac-[Tic²⁷]PYY(22-36): N-alpha-Ac-A S L R H [Tic] L N L V T R Q R Y-NH₂ (SEQ. ID No. 42); N-alpha-Ac-[Phe²⁷]PYY(25-36): N-alpha-Ac-H [Phe] L N L V T R Q R Y-NH₂ (SEQ. ID No. 43); N-alpha-Ac-[Phe²⁷,Thi³⁶]PYY(22-36): N-alpha-Ac-A S L R H (Phel L N L V T R Q R [Thi]-NH₂ (SEQ. ID No. 44); N-alpha-Ac-[Thz²⁶, Phe²⁷]PYY(22-36): N-alpha-Ac-A S L R [Thz][Phe] L N L V T R q R Y-NH₂ (SEQ. ID No. 45); N-alpha-Ac.[Pcp²⁷]PYY(22-36): N-alpha-Ac-A S L R H [Pcp] L N L V T R Q R Y-NH₂ (SEQ. ID No. 46); N-alpha-Ac-[Ph^{22,27}]PYY(22-36): N-alpha-Ac-[Phe]S L R N [Phe] L N L V T R Q R Y-NH₂ (SEQ. ID No. 47); N-alpha-Ac-[Tyr²², Phe²⁷] PYY(22-36): N-alpha-Ac-[Tyr] S L R H [Phe] L N L V T R Q R Y-NH² (SEQ. ID No. 48); N-alpha-Ac-[Trp²⁸]PYY(22-36): N-alpha-Ac-A S L R H Y [Trp] N L V T R Q R Y-NH₂ (SEQ. ID No. 49); N-alpha-Ac-[Trp²⁸]PYY(22-36): N-alpha-Ac-A S L R H Y L N [Trp] V T R Q R Y-NH₂ (SEQ. ID No. 50); N-alpha-Ac-[Ala.sup.26, Phe²⁷]PYY(22-36): N-alpha-Ac-A S L R [Ala] [Phe] L N L V T R Q R Y-NH₂ (SEQ. ID No. 51); N-alpha-Ac-[Bth²⁷]PYY(22-36): N-alpha-Ac-A S L R H [Bth] L N L V T R Q R Y-NH₂ (SEQ. ID No. 52); N-alpha-Ac-[Phe²⁷]PYY(22-36): N-alpha-Ac-A S L R H [Phe] L N L V T R Q R Y-NH₂ (SEQ. ID No. 53); N-alpha-Ac-[Phe^{27,36}]PYY(22-36): N-alpha-Ac-A S L R H [Phe] L N L V T R Q R [Phe]-NH₂ (SEQ. ID No. 54); N-alpha-Ac-[Phe²⁷, D-TrP³²]PYY(22-36): N-alpha-Ac-A S L R H [Phe] L N L V [D-Trp] R Q R Y-NH₂ (SEQ. ID No. 55).

Additional analogs are described in Balasubramaniam, et al., Peptide Research 1:32 (1988); Japanese Patent Application No. 2,225,497 (1990); Balasubramaniam, et al., Peptides 14:1011, 1993; Grandt, et al., Reg. Peptides 51:151 (1994); PCT International Application No. 94/03380.

Balasubramaniam, et al. describes analogs PYY(1-28); PYY(1-22); PYY(22-28); PYY(22-36); and PYY(27-36). Grandt, et al. discusses PYY(1-36) (SEQ ID NO: 1) and PYY(3-36). The above described peptides typically bind to the Y receptors in the brain and elsewhere, especially the Y2 and/or Y5 receptors. Typically these peptides are synthesized in endotoxin-free or pyrogen-free forms although this is not always necessary.

PYY agonists include rat PYY: Tyr Pro Ala Lys Pro Glu Ala Pro Gly Glu Asp Ala Ser Pro Glu Glu Leu Ser Arg Tyr Tyr Ala Ser Leu Arg His Tyr Leu Asn Leu Val Thr Arg Gln Arg Tyr (SEQ ID NO: 56) and the amino terminus truncated forms corresponding to the human; pig PYY: Tyr Pro Ala Lys Pro Glu Ala Pro Gly Glu Asp Ala Ser Pro Glu Glu Leu Ser Arg Tyr Tyr Ala Ser Leu Arg His Tyr Leu Asn Leu Val Thr Arg Gln Arg Tyr (SEQ ID NO: 57) and the amino terminus truncated forms corresponding to the human; and guinea pig PYY: Tyr Pro Ser Lys Pro Glu Ala Pro Gly Ser Asp Ala Ser Pro Glu Glu Leu Ala Arg Tyr Tyr Ala Ser Leu Arg His Tyr Leu Asn Leu Val Thr Arg Gln Arg Tyr (SEQ ID NO: 58) and the amino terminus truncated forms corresponding to the human.

According to the present invention a PYY peptide also includes the free bases, acid addition salts or metal salts, such as potassium or sodium salts of the peptides, and PYY peptides that have been modified by such processes as amidation, glycosylation, acylation, sulfation, phosphorylation, acetylation, cyclization and other well known covalent modification methods. These peptides typically bind to the Y receptors in the brain and elsewhere, especially the Y2 and/or Y5 receptors. Typically these peptides are synthesized in endotoxin-free or pyrogen-free forms although this is not always necessary.

### Neuropeptide Y Agonists

NPY is another Y2 receptor-binding peptide. NPY peptides include full-length human NPY(1-36): Tyr Pro Ser Lys Pro Asp Asn Pro Gly Glu Asp Ala Pro Ala Glu Asp Met Ala Arg Tyr Tyr Ser Ala Leu Arg His Tyr Ile Asn Leu Ile Thr Arg Gln Arg Tyr (SEQ ID NO: 59) as well as well as fragments of NPY(1-36), which have been truncated at the amino terminus. To be effective in binding the Y2 receptor, the NPY agonist should have at least the last 11 amino acid residues at the carboxyl terminus, i.e., be comprised of NPY(26-36). Other examples of NPY agonists that bind to the Y2 receptor are NPY(3-36), NPY(4-36), NPY(5-36), NPY(6-36), NPY(7-36), NPY(8-36), NPY(9-36), NPY(10-36), NPY(11-36), NPY(12-36), NPY(13-36), NPY(14-36), NPY(15-36), NPY(16-36), NPY(17-36), NPY(18-36), NPY(19-36), NPY(20-36), NPY(21-36), NPY(22-36), NPY(23-36), NPY(24-36), and NPY(25-36).

Other NPY agonists include rat NPY: Tyr Pro Ser Lys Pro Asp Asn Pro Gly Glu Asp Ala Pro Ala Glu Asp Met Ala Arg Tyr Tyr Ser Ala Leu Arg His Tyr Ile Asn Leu Ile Thr Arg Gln Arg Tyr (SEQ ID NO: 60) and the amino terminus truncated forms from NPY(3-36) to NPY(26-36) as in the human form; rabbit NPY: Tyr Pro Ser Lys Pro Asp Asn Pro Gly Glu Asp Ala Pro Ala Glu Asp Met Ala Arg Tyr Tyr Ser Ala Leu Arg His Tyr Ile Asn Leu Ile Thr Arg Gln Arg Tyr (SEQ ID NO: 61) and the amino terminus truncated forms from NPY(3-36) to NPY(26-36) as in the human form; dog NPY: Tyr Pro Ser Lys Pro Asp Asn Pro Gly Glu Asp Ala Pro Ala Glu Asp Met Ala Arg Tyr Tyr Ser Ala Leu Arg His Tyr Ile Asn Leu Ile Thr Arg Gln Arg Tyr (SEQ ID NO: 62) and the amino terminus truncated forms NPY(3-36) to NPY(26-36) as in the human form; pig NPY: Tyr Pro Ser Lys Pro Asp Asn Pro Gly Glu Asp Ala Pro Ala Glu Asp Leu Ala Arg Tyr Tyr Ser Ala Leu Arg His Tyr Ile Asn Leu Ile Thr Arg Gln Arg Tyr (SEQ ID NO: 63) and the amino terminus truncated forms from NPY(3-36) to NPY(26-36) as in the human form; cow NPY: Tyr Pro Ser Lys Pro Asp Asn Pro Gly Glu Asp Ala Pro Ala Glu Asp Leu Ala Arg Tyr Tyr Ser Ala Leu Arg His Tyr Ile Asn Leu Ile Thr Arg Gln Arg Tyr (SEQ ID NO: 64) and the amino terminus truncated forms from NPY(3-36) to NPY(26-36) as in the human form; sheep NPY: Tyr Pro Ser Lys Pro Asp Asn Pro Gly Asp Asp Ala Pro Ala Glu Asp Leu Ala Arg Tyr Tyr Ser Ala Leu Arg His Tyr Ile Asn Leu Ile Thr Arg Gln Arg Tyr (SEQ ID NO: 65) and the amino terminus truncated forms from NPY(3-36) to NPY(26-36) as in the human form; and guinea pig NPY: Tyr Pro Ser Lys Pro Asp Asn Pro Gly Glu Asp Ala Pro Ala Glu Asp Met Ala Arg Tyr Tyr Ser Ala Leu Arg His Tyr Ile Asn Leu Ile Thr Arg Gln Arg Tyr (SEQ ID NO: 66) and the amino terminus truncated forms from NPY(3-36) to NPY(26-36) as in the human form. According to the present invention a NPY peptide also includes the free bases, acid addition salts or metal salts, such as potassium or sodium salts of the peptides, and NPY peptides that have been modified by such processes as amidation, glycosylation, acylation, sulfation, phosphorylation, acetylation, cyclization and other known covalent modification methods. These peptides typically bind to the Y receptors in the brain and elsewhere, especially the Y2 and/or Y5 receptors. Typically these peptides are synthesized in endotoxin-free or pyrogen-free forms although this is not always necessary.

### Pancreatic Peptide

Pancreatic Peptide (PP) and PP agonist also bind to the Y2 receptor. Examples of the PP agonists are the full-length human PP(1-36): Ala Ser Leu Glu Pro Glu Tyr Pro Gly Asp Asn Ala Thr Pro Glu Gln Met Ala Gln Tyr Ala Ala Glu Leu Arg Arg Tyr Ile Asn Met Leu Thr Arg Pro Arg Tyr (SEQ ID NO: 67) and a number of PP fragments, which are truncated at the amino-terminus. To bind to the Y2 receptor the PP agonist must have the last 11 amino acid residues at the carboxyl-terminus, PP(26-36). Examples of other PP, which bind to the Y2 receptor, are PP(3-36), PP(4-36), PP(5-36), PP(6-36), PP(7-36), PP(8-36), PP(9-36), PP(10-36), PP(11-36), PP(12-36), PP(13-36), PP(14-36), PP(15-36), PP(16-36), PP(17-36), PP(18-36), PP(19-36), PP(20-36), PP(21-36), PP(22-36), PP(23-36), PP(24-36), and PP(25-36).

Other PP agonists include sheep PP: Ala Pro Leu Glu Pro Val Tyr Pro Gly Asp Asn Ala Thr Pro Glu Gln Met Ala Gln Tyr Ala Ala Asp Leu Arg Arg Tyr Ile Asn Met Leu Thr Arg Pro Arg Tyr (SEQ ID NO: 68) and the amino terminus truncated forms from PP(3-36) to PP(26-36) as in the human form; pig PP: Ala Pro Leu Glu Pro Val Tyr Pro Gly Asp Asp Ala Thr Pro Glu Met Ala Gln Tyr Ala Ala Glu Leu Arg Arg Tyr Ile Asn Met Leu Thr Arg Pro Arg Tyr (SEQ ID NO: 69) and the amino terminus truncated forms from PP(3-36) to PP(26-36) as in the human form; dog PP: Ala Pro Leu Glu Pro Val Tyr Pro Gly Asp Asp Ala Thr Pro Glu Gln Met Ala Gln Tyr Ala Ala Glu Leu Arg Arg Tyr Ile Asn Met Leu Thr Arg Pro Arg Tyr (SEQ ID NO: 70) and the amino terminus truncated forms PP(3-36) to PP(26-36) as in the human form; cat PP: Ala Pro Leu Glu Pro Val Tyr Pro Gly Asp Asn Ala Thr Pro Glu Gln Met Ala Gln Tyr Ala Ala Glu Leu Arg Arg Tyr Ile Asn Met Leu Thr Arg Pro Arg Tyr (SEQ ID NO: 71) and the amino terminus truncated forms from PP(3-36) to PP(26-36) as in the human form; cow PP: Ala Pro Leu Glu Pro Glu Tyr Pro Gly Asp Asp Ala Thr Pro Glu Gln Met Ala Gln Tyr Ala Ala Glu Leu Arg Arg Tyr Ile Asn Met Leu Thr Arg Pro Arg Tyr (SEQ ID NO: 72) and the amino terminus truncated forms from PP(3-36) to PP(26-36) as in the human form; rat PP: Ala Pro Leu Glu Pro Met Tyr Pro Gly Asp Tyr Ala Thr His Glu Gln Arg Ala Gln Tyr Glu Thr Gln Leu Arg Arg Tyr Ile Asn Thr Leu Thr Arg Pro Arg Tyr (SEQ ID NO: 73) and the amino terminus truncated forms from PP(3-36) to PP(26-36) as in the human form; mouse PP: Ala Pro Leu Glu Pro Met Tyr Pro Gly Asp Tyr Ala Thr His Glu Gln Arg Ala Gln Tyr Glu Thr Gln Leu Arg Arg Tyr Ile Asn Thr Leu Thr Arg Pro Arg Tyr (SEQ ID NO: 74) and the amino terminus truncated forms from PP(3-36) to PP(26-36) as in the human form; and guinea pig PP: Ala Pro Leu Glu Pro Met Tyr Pro Gly Asp Tyr Ala Thr Pro Glu Gln Met Ala Gln Tyr Glu Thr Gln Leu Arg Arg Tyr Ile Asn Thr Leu Thr Arg Pro Arg Tyr (SEQ ID NO: 75) and the amino terminus truncated forms from PP(3-36) to PP(26-36) as in the human form.

According to the present invention a PP peptide also includes the free bases, acid addition salts or metal salts, such as potassium or sodium salts of the peptides, and PP peptides that have been modified by such processes as amidation, glycosylation, acylation, sulfation, phosphorylation, acetylation, cyclization, and other known covalent modification methods. These peptides typically bind to the Y receptors in the brain and elsewhere, especially the Y2 and/or Y5 receptors. Typically these peptides are synthesized in endotoxin-free or pyrogen-free forms although this is not always necessary.

### Peptide and Protein Analogs and Mimetics

Included within the definition of biologically active peptides and proteins for use within the invention are natural or synthetic, therapeutically or prophylactically active, peptides (comprised of two or more covalently linked amino acids), proteins, peptide or protein fragments, peptide or protein analogs, and chemically modified derivatives or salts of active peptides or proteins. A wide variety of useful analogs and mimetics of Y2 receptor-binding peptide are contemplated for use within the invention and can be produced and tested for biological activity according to known methods. Often, the peptides or proteins of Y2 receptor-binding peptide or other biologically active peptides or proteins for use within the invention are muteins that are readily obtainable by partial substitution, addition, or deletion of amino acids within a naturally occurring or native (e.g., wild-type, naturally occurring mutant, or allelic variant) peptide or protein sequence. Additionally, biologically active fragments of native peptides or proteins are included. Such mutant derivatives and fragments substantially retain the desired biological activity of the native peptide or proteins. In the case of peptides or proteins having carbohydrate chains, biologically active variants marked by alterations in these carbohydrate species are also included within the invention.

As used herein, the term “conservative amino acid substitution” refers to the general interchangeability of amino acid residues having similar side chains. For example, a commonly interchangeable group of amino acids having aliphatic side chains is alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Examples of conservative substitutions include the substitution of a non-polar (hydrophobic) residue such as isoleucine, valine, leucine or methionine for another. Likewise, the present invention contemplates the substitution of a polar (hydrophilic) residue such as between arginine and lysine, between glutamine and asparagine, and between threonine and serine. Additionally, the substitution of a basic residue such as lysine, arginine or histidine for another or the substitution of an acidic residue such as aspartic acid or glutamic acid for another is also contemplated. Exemplary conservative amino acids substitution groups are: valine-leucine-isoleucine, phenylalanine-tyrosine, lysine-arginine, alanine-valine, and asparagine-glutamine. By aligning a peptide or protein analog optimally with a corresponding native peptide or protein, and by using appropriate assays, e.g., adhesion protein or receptor binding assays, to determine a selected biological activity, one can readily identify operable peptide and protein analogs for use within the methods and compositions of the invention. Operable peptide and protein analogs are typically specifically immunoreactive with antibodies raised to the corresponding native peptide or protein.

### Pharmacokinetic (PK) Parameters

As used herein “peak concentration (Cₘₐₓ) of Y2 receptor-binding peptide in a blood plasma”, “area under concentration vs. time curve (AUC) of Y2 receptor-binding peptide in a blood plasma”, “time to maximal plasma concentration (tₘₐₓ) of Y2 receptor-binding peptide in a blood plasma” are pharmacokinetic parameters known to one skilled in the art. Laursen et al., Eur. J. Endocrinology 135:309-315, 1996. The “concentration vs. time curve” measures the concentration of Y2 receptor-binding peptide in a blood serum of a subject vs. time after administration of a dosage of Y2 receptor-binding peptide to the subject either by intranasal, intramuscular, subcutaneous, or other parenteral route of administration. “Cₘₐₓ” is the maximum concentration of Y2 receptor-binding peptide in the blood serum of a subject following a single dosage of Y2 receptor-binding peptide to the subject. “tₘₐₓ” is the time to reach maximum concentration of Y2 receptor-binding peptide in a blood serum of a subject following administration of a single dosage of Y2 receptor-binding peptide to the subject.

As used herein, “area under concentration vs. time curve (AUC) of Y2 receptor-binding peptide in a blood plasma” is calculated according to the linear trapezoidal rule and with addition of the residual areas. A decrease of 23% or an increase of 30% between two dosages would be detected with a probability of 90% (type II error β=10%). The “delivery rate” or “rate of absorption” is estimated by comparison of the time (tₘₐₓ) to reach the maximum concentration (Cₘₐₓ). Both Cₘₐₓ and tₘₐₓ are analyzed using non-parametric methods. Comparisons of the pharmacokinetics of intramuscular, subcutaneous, intravenous and intranasal Y2 receptor-binding peptide administrations were performed by analysis of variance (ANOVA). For pair wise comparisons a Bonferroni-Holmes sequential procedure was used to evaluate significance. The dose-response relationship between the three nasal doses was estimated by regression analysis. P<0.05 was considered significant. Results are given as mean values+/−SEM.

While the mechanism of absorption promotion may vary with different mucosal delivery-enhancing agents of the invention, useful reagents in this context will not substantially adversely affect the mucosal tissue and will be selected according to the physicochemical characteristics of the particular Y2 receptor-binding peptide or other active or delivery-enhancing agent. In this context, delivery-enhancing agents that increase penetration or permeability of mucosal tissues will often result in some alteration of the protective permeability barrier of the mucosa. For such delivery-enhancing agents to be of value within the invention, it is generally desired that any significant changes in permeability of the mucosa be reversible within a time frame appropriate to the desired duration of drug delivery. Furthermore, there should be no substantial, cumulative toxicity, nor any permanent deleterious changes induced in the barrier properties of the mucosa with long-term use.

### Stability

An approach for stabilizing solid protein formulations of the invention is to increase the physical stability of purified, e.g., lyophilized, protein. This will inhibit aggregation via hydrophobic interactions as well as via covalent pathways that may increase as proteins unfold. Stabilizing formulations in this context often include polymer-based formulations, for example a biodegradable hydrogel formulation/delivery system. As noted above, the critical role of water in protein structure, function, and stability is well known. Typically, proteins are relatively stable in the solid state with bulk water removed. However, solid therapeutic protein formulations may become hydrated upon storage at elevated humidity or during delivery from a sustained release composition or device. The stability of proteins generally drops with increasing hydration. Water can also play a significant role in solid protein aggregation, for example, by increasing protein flexibility resulting in enhanced accessibility of reactive groups, by providing a mobile phase for reactants, and by serving as a reactant in several deleterious processes such as beta-elimination and hydrolysis.

Protein preparations containing between about 6% to 28% water are the most unstable. Below this level, the mobility of bound water and protein internal motions are low. Above this level, water mobility and protein motions approach those of full hydration. Up to a point, increased susceptibility toward solid-phase aggregation with increasing hydration has been observed in several systems. However, at higher water content, less aggregation is observed because of the dilution effect.

In accordance with these principles, an effective method for stabilizing peptides and proteins against solid-state aggregation for mucosal delivery is to control the water content in a solid formulation and maintain the water activity in the formulation at optimal levels. This level depends on the nature of the protein, but in general, proteins maintained below their “monolayer” water coverage will exhibit superior solid-state stability.

A variety of additives, diluents, bases and delivery vehicles are provided within the invention that effectively controls water content to enhance protein stability. These reagents and carrier materials effective as anti-aggregation agents in this sense include, for example, polymers of various functionalities, such as polyethylene glycol, dextran, diethylaminoethyl dextran, and carboxymethyl cellulose, which significantly increase the stability and reduce the solid-phase aggregation of peptides and proteins admixed therewith or linked thereto

Certain additives also impart significant physical stability to dry, e.g., lyophilized proteins. These additives can also be used within the invention to protect the proteins against aggregation not only during lyophilization but also during storage in the dry state.

Various additional preparative components and methods, as well as specific formulation additives, are provided herein which yield formulations for mucosal delivery of aggregation-prone peptides and proteins, in which the peptide or protein is stabilized in a substantially pure, unaggregated form using a solubilization agent. A range of components and additives are contemplated for use within these methods and formulations. Exemplary of these solubilization agents are cyclodextrins (CDs), which selectively bind hydrophobic side chains of polypeptides. These CDs have been found to bind to hydrophobic patches of proteins in a manner that significantly inhibits aggregation. This inhibition is selective with respect to both the CD and the protein involved. Such selective inhibition of protein aggregation provides additional advantages within the intranasal delivery methods and compositions of the invention. Additional agents for use in this context include CD dimers, trimers and tetramers with varying geometries controlled by the linkers that specifically block aggregation of peptides and protein. Yet solubilization agents and methods for incorporation within the invention involve the use of peptides and peptide mimetics to selectively block protein-protein interactions. In one aspect, the specific binding of hydrophobic side chains reported for CD multimers is extended to proteins via the use of peptides and peptide mimetics that similarly block protein aggregation. A wide range of suitable methods and anti-aggregation agents are available for incorporation within the compositions and procedures of the invention.

### Proteinase Inhibitors

Another excipient that may be included in a trans-mucosal preparation is a degradative enzyme inhibitor. Exemplary mucoadhesive polymer-enzyme inhibitor complexes that are useful within the mucosal delivery formulations and methods of the invention include, but are not limited to: Carboxymethylcellulose-pepstatin (with anti-pepsin activity); Poly(acrylic acid)-Bowman-Birk inhibitor (anti-chymotrypsin); Poly(acrylic acid)-chymostatin (anti-chymotrypsin); Poly(acrylic acid)-elastatinal (anti-elastase); Carboxymethylcellulose-elastatinal (anti-elastase); Polycarbophil-elastatinal (anti-elastase); Chitosan-antipain (anti-trypsin); Poly(acrylic acid)-bacitracin (anti-aminopeptidase N); Chitosan-EDTA (anti-aminopeptidase N, anti-carboxypeptidase A); Chitosan-EDTA-antipain (anti-trypsin, anti-chymotrypsin, anti-elastase). As described in further detail below, certain embodiments of the invention will optionally incorporate a novel chitosan derivative or chemically modified form of chitosan. One such novel derivative for use within the invention is denoted as a β-[1→4]-2-guanidino-2-deoxy-D-glucose polymer (poly-GuD).

Any inhibitor that inhibits the activity of an enzyme to protect the biologically active agent(s) may be usefully employed in the compositions and methods of the invention. Useful enzyme inhibitors for the protection of biologically active proteins and peptides include, for example, soybean trypsin inhibitor, pancreatic trypsin inhibitor, chymotrypsin inhibitor and trypsin and chrymotrypsin inhibitor isolated from potato (solanum tuberosum L.) tubers. A combination or mixtures of inhibitors may be employed. Additional inhibitors of proteolytic enzymes for use within the invention include ovomucoid-enzyme, gabaxate mesylate, alpha1-antitrypsin, aprotinin, amastatin, bestatin, puromycin, bacitracin, leupepsin, alpha2-macroglobulin, pepstatin and egg white or soybean trypsin inhibitor. These and other inhibitors can be used alone or in combination. The inhibitor(s) may be incorporated in or bound to a carrier, e.g., a hydrophilic polymer, coated on the surface of the dosage form which is to contact the nasal mucosa, or incorporated in the superficial phase of the surface, in combination with the biologically active agent or in a separately administered (e.g., pre-administered) formulation.

The amount of the inhibitor, e.g., of a proteolytic enzyme inhibitor that is optionally incorporated in the compositions of the invention will vary depending on (a) the properties of the specific inhibitor, (b) the number of functional groups present in the molecule (which may be reacted to introduce ethylenic unsaturation necessary for copolymerization with hydrogel forming monomers), and (c) the number of lectin groups, such as glycosides, which are present in the inhibitor molecule. It may also depend on the specific therapeutic agent that is intended to be administered. Generally speaking, a useful amount of an enzyme inhibitor is from about 0.1 mg/ml to about 50 mg/ml, often from about 0.2 mg/ml to about 25 mg/ml, and more commonly from about 0.5 mg/ml to 5 mg/ml of the of the formulation (i.e., a separate protease inhibitor formulation or combined formulation with the inhibitor and biologically active agent).

In the case of trypsin inhibition, suitable inhibitors may be selected from, e.g., aprotinin, BBI, soybean trypsin inhibitor, chicken ovomucoid, chicken ovoinhibitor, human pancreatic trypsin inhibitor, camostat mesilate, flavonoid inhibitors, antipain, leupeptin, p-aminobenzamidine, AEBSF, TLCK (tosyllysine chloromethylketone), APMSF, DFP, PMSF, and poly(acrylate) derivatives. In the case of chymotrypsin inhibition, suitable inhibitors may be selected from, e.g., aprotinin, BBI, soybean trypsin inhibitor, chymostatin, benzyloxycarbonyl-Pro-Phe-CHO, FK-448, chicken ovoinhibitor, sugar biphenylboronic acids complexes, DFP, PMSF, β-phenylpropionate, and poly(acrylate) derivatives. In the case of elastase inhibition, suitable inhibitors may be selected from, e.g., elastatinal, methoxysuccinyl-Ala-Ala-Pro-Val-chloromethylketone (SEQ ID NO. 76) (MPCMK), BBI, soybean trypsin inhibitor, chicken ovoinhibitor, DFP, and PMSF.

Additional enzyme inhibitors for use within the invention are selected from a wide range of non-protein inhibitors that vary in their degree of potency and toxicity. As described in further detail below, immobilization of these adjunct agents to matrices or other delivery vehicles, or development of chemically modified analogs, may be readily implemented to reduce or even eliminate toxic effects, when they are encountered. Among this broad group of candidate enzyme inhibitors for use within the invention are organophosphorous inhibitors, such as diisopropylfluorophosphate (DFP) and phenylmethylsulfonyl fluoride (PMSF), which are potent, irreversible inhibitors of serine proteases (e.g., trypsin and chymotrypsin). The additional inhibition of acetylcholinesterase by these compounds makes them highly toxic in uncontrolled delivery settings. Another candidate inhibitor, 4-(2-Aminoethyl)-benzenesulfonyl fluoride (AEBSF), has an inhibitory activity comparable to DFP and PMSF, but it is markedly less toxic. (4-Aminophenyl)-methanesulfonyl fluoride hydrochloride (APMSF) is another potent inhibitor of trypsin, but is toxic in uncontrolled settings. In contrast to these inhibitors, 4-(4-isopropylpiperadinocarbonyl)phenyl 1,2,3,4,-tetrahydro-1-naphthoate methanesulphonate (FK-448) is a low toxic substance, representing a potent and specific inhibitor of chymotrypsin. Further representatives of this non-protein group of inhibitor candidates, and also exhibiting low toxic risk, are camostat mesilate (N,N′-dimethyl carbamoylmethyl-p-(p′-guanidino-benzoyloxy)phenylacetate methane-sulphonate).

Yet another type of enzyme inhibitory agent for use within the methods and compositions of the invention are amino acids and modified amino acids that interfere with enzymatic degradation of specific therapeutic compounds. For use in this context, amino acids and modified amino acids are substantially non-toxic and can be produced at a low cost. However, due to their low molecular size and good solubility, they are readily diluted and absorbed in mucosal environments. Nevertheless, under proper conditions, amino acids can act as reversible, competitive inhibitors of protease enzymes. Certain modified amino acids can display a much stronger inhibitory activity. A desired modified amino acid in this context is known as a ‘transition-state’ inhibitor. The strong inhibitory activity of these compounds is based on their structural similarity to a substrate in its transition-state geometry, while they are generally selected to have a much higher affinity for the active site of an enzyme than the substrate itself. Transition-state inhibitors are reversible, competitive inhibitors. Examples of this type of inhibitor are α-aminoboronic acid derivatives, such as boro-leucine, boro-valine and boro-alanine. The boron atom in these derivatives can form a tetrahedral boronate ion that is believed to resemble the transition state of peptides during their hydrolysis by aminopeptidases. These amino acid derivatives are potent and reversible inhibitors of aminopeptidases and it is reported that boro-leucine is more than 100-times more effective in enzyme inhibition than bestatin and more than 1000-times more effective than puromycin. Another modified amino acid for which a strong protease inhibitory activity has been reported is N-acetylcysteine, which inhibits enzymatic activity of aminopeptidase N. This adjunct agent also displays mucolytic properties that can be employed within the methods and compositions of the invention to reduce the effects of the mucus diffusion barrier.

Still other useful enzyme inhibitors for use within the coordinate administration methods and combinatorial formulations of the invention may be selected from peptides and modified peptide enzyme inhibitors. An important representative of this class of inhibitors is the cyclic dodecapeptide, bacitracin, obtained from Bacillus licheniformis. In addition to these types of peptides, certain dipeptides and tripeptides display weak, non-specific inhibitory activity towards some protease. By analogy with amino acids, their inhibitory activity can be improved by chemical modifications. For example, phosphinic acid dipeptide analogs are also ‘transition-state’ inhibitors with a strong inhibitory activity towards aminopeptidases. They have reportedly been used to stabilize nasally administered leucine enkephalin. Another example of a transition-state analog is the modified pentapeptide pepstatin, which is a very potent inhibitor of pepsin. Structural analysis of pepstatin, by testing the inhibitory activity of several synthetic analogs, demonstrated the major structure-function characteristics of the molecule responsible for the inhibitory activity. Another special type of modified peptide includes inhibitors with a terminally located aldehyde function in their structure. For example, the sequence benzyloxycarbonyl-Pro-Phe-CHO, which fulfills the known primary and secondary specificity requirements of chymotrypsin, has been found to be a potent reversible inhibitor of this target proteinase. The chemical structures of further inhibitors with a terminally located aldehyde function, e.g. antipain, leupeptin, chymostatin and elastatinal, are also known in the art, as are the structures of other known, reversible, modified peptide inhibitors, such as phosphoramidon, bestatin, puromycin and amastatin.

Due to their comparably high molecular mass, polypeptide protease inhibitors are more amenable than smaller compounds to concentrated delivery in a drug-carrier matrix. Additional agents for protease inhibition within the formulations and methods of the invention involve the use of complexing agents. These agents mediate enzyme inhibition by depriving the intranasal environment (or preparative or therapeutic composition) of divalent cations, which are co-factors for many proteases. For instance, the complexing agents EDTA and DTPA as coordinately administered or combinatorially formulated adjunct agents, in suitable concentration, will be sufficient to inhibit selected proteases to thereby enhance intranasal delivery of biologically active agents according to the invention. Further representatives of this class of inhibitory agents are EGTA, 1,10-phenanthroline and hydroxychinoline. In addition, due to their propensity to chelate divalent cations, these and other complexing agents are useful within the invention as direct, absorption-promoting agents.

As noted in more detail elsewhere herein, it is also contemplated to use various polymers, particularly mucoadhesive polymers, as enzyme inhibiting agents within the coordinate administration, multi-processing and/or combinatorial formulation methods and compositions of the invention. For example, poly(acrylate) derivatives, such as poly(acrylic acid) and polycarbophil, can affect the activity of various proteases, including trypsin, chymotrypsin. The inhibitory effect of these polymers may also be based on the complexation of divalent cations such as Ca²⁺ and Zn²⁺. It is further contemplated that these polymers may serve as conjugate partners or carriers for additional enzyme inhibitory agents, as described above. For example, a chitosan-EDTA conjugate has been developed and is useful within the invention that exhibits a strong inhibitory effect towards the enzymatic activity of zinc-dependent proteases. The mucoadhesive properties of polymers following covalent attachment of other enzyme inhibitors in this context are not expected to be substantially compromised, nor is the general utility of such polymers as a delivery vehicle for biologically active agents within the invention expected to be diminished. On the contrary, the reduced distance between the delivery vehicle and mucosal surface afforded by the mucoadhesive mechanism will minimize presystemic metabolism of the active agent, while the covalently bound enzyme inhibitors remain concentrated at the site of drug delivery, minimizing undesired dilution effects of inhibitors as well as toxic and other side effects caused thereby. In this manner, the effective amount of a coordinately administered enzyme inhibitor can be reduced due to the exclusion of dilution effects.

Exemplary mucoadhesive polymer-enzyme inhibitor complexes that are useful within the mucosal formulations and methods of the invention include, but are not limited to: Carboxymethylcellulose-pepstatin (with anti-pepsin activity); Poly(acrylic acid)-Bowman-Birk inhibitor (anti-chymotrypsin); Poly(acrylic acid)-chymostatin (anti-chymotrypsin); Poly(acrylic acid)-elastatinal (anti-elastase); Carboxymethylcellulose-elastatinal (anti-elastase); Polycarbophil-elastatinal (anti-elastase); Chitosan-antipain (anti-trypsin); Poly(acrylic acid)-bacitracin (anti-aminopeptidase N); Chitosan-EDTA (anti-aminopeptidase N, anti-carboxypeptidase A); Chitosan-EDTA-antipain (anti-trypsin, anti-chymotrypsin, anti-elastase).

### Mucosal Delivery

Mucosal delivery formulations of the present invention comprise Y2 receptor-binding peptide, analogs and mimetics, typically combined together with one or more pharmaceutically acceptable carriers and, optionally, other therapeutic ingredients. The carrier(s) must be “pharmaceutically acceptable” in the sense of being compatible with the other ingredients of the formulation and not eliciting an unacceptable deleterious effect in the subject. Such carriers are described herein above or are otherwise well known to those skilled in the art of pharmacology. Desirably, the formulation should not include substances such as enzymes or oxidizing agents with which the biologically active agent to be administered is known to be incompatible. The formulations may be prepared by any of the methods well known in the art of pharmacy.

Within the compositions and methods of the invention, the Y2 receptor-binding peptide proteins, analogs and mimetics, and other biologically active agents disclosed herein may be administered to subjects by a variety of mucosal administration modes, including by oral, rectal, vaginal, intranasal, intrapulmonary, or transdermal delivery, or by topical delivery to the eyes, ears, skin or other mucosal surfaces. Optionally, Y2 receptor-binding peptide proteins, analogs and mimetics, and other biologically active agents disclosed herein can be coordinately or adjunctively administered by non-mucosal routes, including by intramuscular, subcutaneous, intravenous, intra-atrial, intra-articular, intraperitoneal, or parenteral routes. In other alternative embodiments, the biologically active agent(s) can be administered ex vivo by direct exposure to cells, tissues or organs originating from a mammalian subject, for example as a component of an ex vivo tissue or organ treatment formulation that contains the biologically active agent in a suitable, liquid or solid carrier.

Compositions according to the present invention are often administered in an aqueous solution as a nasal or pulmonary spray and may be dispensed in spray form by a variety of methods known to those skilled in the art. Preferred systems for dispensing liquids as a nasal spray are disclosed in U.S. Pat. No. 4,511,069. The formulations may be presented in multi-dose containers, for example in the sealed dispensing system disclosed in U.S. Pat. No. 4,511,069. Additional aerosol delivery forms may include, e.g., compressed air-, jet-, ultrasonic-, and piezoelectric nebulizers, which deliver the biologically active agent dissolved or suspended in a pharmaceutical solvent, e.g., water, ethanol, or a mixture thereof.

Nasal and pulmonary spray solutions of the present invention typically comprise the drug or drug to be delivered, optionally formulated with a surface-active agent, such as a nonionic surfactant (e.g., polysorbate-80), and one or more buffers. In some embodiments of the present invention, the nasal spray solution further comprises a propellant. The pH of the nasal spray solution is optionally between about pH 3.0 and 6.0, preferably 4.5±0.5. Suitable buffers for use within these compositions are as described above or as otherwise known in the art. Other components may be added to enhance or maintain chemical stability, including preservatives, surfactants, dispersants, or gases. Suitable preservatives include, but are not limited to, phenol, methyl paraben, paraben, m-cresol, thiomersal, chlorobutanol, benzylalkonimum chloride, and the like. Suitable surfactants include, but are not limited to, oleic acid, sorbitan trioleate, polysorbates, lecithin, phosphatidyl cholines, and various long chain diglycerides and phospholipids. Suitable dispersants include, but are not limited to, ethylenediaminetetraacetic acid, and the like. Suitable gases include, but are not limited to, nitrogen, helium, chlorofluorocarbons (CFCs), hydrofluorocarbons (HFCs), carbon dioxide, air, and the like.

Within alternate embodiments, mucosal formulations are administered as dry powder formulations comprising the biologically active agent in a dry, usually lyophilized, form of an appropriate particle size, or within an appropriate particle size range, for intranasal delivery. Minimum particle size appropriate for deposition within the nasal or pulmonary passages is often about 0.5μ mass median equivalent aerodynamic diameter (MMEAD), commonly about 1μ MMEAD, and more typically about 2μ MMEAD. Maximum particle size appropriate for deposition within the nasal passages is often about 10μ MMEAD, commonly about 8μ MMEAD, and more typically about 4μ MMEAD. Intranasally respirable powders within these size ranges can be produced by a variety of conventional techniques, such as jet milling, spray drying, solvent precipitation, supercritical fluid condensation, and the like. These dry powders of appropriate MMEAD can be administered to a patient via a conventional dry powder inhaler (DPI), which rely on the patient's breath, upon pulmonary or nasal inhalation, to disperse the power into an aerosolized amount. Alternatively, the dry powder may be administered via air-assisted devices that use an external power source to disperse the powder into an aerosolized amount, e.g., a piston pump.

To formulate compositions for mucosal delivery within the present invention, the biologically active agent can be combined with various pharmaceutically acceptable additives, as well as a base or carrier for dispersion of the active agent(s). Desired additives include, but are not limited to, pH control agents, such as arginine, sodium hydroxide, glycine, hydrochloric acid, citric acid, etc. In addition, local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione) can be included. When the composition for mucosal delivery is a liquid, the tonicity of the formulation, as measured with reference to the tonicity of 0.9% (w/v) physiological saline solution taken as unity, is typically adjusted to a value at which no substantial, irreversible tissue damage will be induced in the nasal mucosa at the site of administration. Generally, the tonicity of the solution is adjusted to a value of about ⅓ to 3, more typically ½ to 2, and most often ¾ to 1.7.

The biologically active agent may be dispersed in a base or vehicle, which may comprise a hydrophilic compound having a capacity to disperse the active agent and any desired additives. The base may be selected from a wide range of suitable carriers, including but not limited to, copolymers of polycarboxylic acids or salts thereof, carboxylic anhydrides (e.g., maleic anhydride) with other monomers (e.g., methyl (meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters, etc. can be employed as carriers. Hydrophilic polymers and other carriers can be used alone or in combination, and enhanced structural integrity can be imparted to the carrier by partial crystallization, ionic bonding, crosslinking and the like. The carrier can be provided in a variety of forms, including, fluid or viscous solutions, gels, pastes, powders, microspheres and films for direct application to the nasal mucosa. The use of a selected carrier in this context may result in promotion of absorption of the biologically active agent.

The compositions of the invention may alternatively contain as pharmaceutically acceptable carriers substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. For solid compositions, conventional nontoxic pharmaceutically acceptable carriers can be used which include, for example, pharmaceutical grades of mannitol, lactose, starch, magnesium stearate, sodium saccharin, talcum, magnesium carbonate, and the like.

Therapeutic compositions for administering the biologically active agent can also be formulated as a solution, microemulsion, or other ordered structure suitable for high concentration of active ingredients. The carrier can be a solvent or dispersion medium containing, for example, water, ethanol, polyol (for example, glycerol, propylene glycol, and liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants.

In certain embodiments of the invention, the biologically active agent is administered in a time-release formulation, for example in a composition which includes a slow release polymer. The active agent can be prepared with carriers that will protect against rapid release, for example a controlled release vehicle such as a polymer, microencapsulated delivery system or bioadhesive gel. Prolonged delivery of the active agent, in various compositions of the invention can be brought about by including in the composition agents that delay absorption, for example, aluminum monosterate hydrogels and gelatin. When controlled release formulations of the biologically active agent is desired, controlled release binders suitable for use in accordance with the invention include any biocompatible controlled-release material which is inert to the active agent and which is capable of incorporating the biologically active agent. Numerous such materials are known in the art. Useful controlled-release binders are materials that are metabolized slowly under physiological conditions following their intranasal delivery (e.g., at the nasal mucosal surface, or in the presence of bodily fluids following transmucosal delivery). Appropriate binders include but are not limited to biocompatible polymers and copolymers previously used in the art in sustained release formulations. Such biocompatible compounds are non-toxic and inert to surrounding tissues, and do not trigger significant adverse side effects such as nasal irritation, immune response, inflammation, or the like. They are metabolized into metabolic products that are also biocompatible and easily eliminated from the body.

Sterile solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders, methods of preparation include vacuum drying and freeze-drying which yields a powder of the active ingredient plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

Mucosal administration according to the invention allows effective self-administration of treatment by patients, provided that sufficient safeguards are in place to control and monitor dosing and side effects. Mucosal administration also overcomes certain drawbacks of other administration forms, such as injections, that are painful and expose the patient to possible infections and may present drug bioavailability problems. For nasal and pulmonary delivery, systems for controlled aerosol dispensing of therapeutic liquids as a spray are well known. In one embodiment, metered doses of active agent are delivered by means of a specially constructed mechanical pump valve, U.S. Pat. No. 4,511,069.

### Dosage

For prophylactic and treatment purposes, the biologically active agent(s) disclosed herein may be administered to the subject in a single bolus delivery, or in a repeated administration protocol (e.g., by an hourly, daily or weekly, repeated administration protocol). In this context, a therapeutically effective dosage of PYY may include repeated doses within a prolonged prophylaxis or treatment regimen that will yield clinically significant results to alleviate one or more symptoms or detectable conditions associated with a targeted disease or condition as set forth above. Determination of effective dosages in this context is typically based on animal model studies followed up by human clinical trials and is guided by determining effective dosages and administration protocols that significantly reduce the occurrence or severity of targeted disease symptoms or conditions in the subject. Suitable models in this regard include, for example, murine, rat, porcine, feline, non-human primate, and other accepted animal model subjects known in the art. Alternatively, effective dosages can be determined using in vitro models (e.g., immunologic and histopathologic assays). Using such models, only ordinary calculations and adjustments are typically required to determine an appropriate concentration and dose to administer a therapeutically effective amount of the biologically active agent(s) (e.g., amounts that are intranasally effective, transdermally effective, intravenously effective, or intramuscularly effective to elicit a desired response).

The actual dosage of biologically active agents will of course vary according to factors such as the disease indication and particular status of the subject (e.g., the subject's age, size, fitness, extent of symptoms, susceptibility factors, etc), time and route of administration, other drugs or treatments being administered concurrently, as well as the specific pharmacology of the biologically active agent(s) for eliciting the desired activity or biological response in the subject. Dosage regimens may be adjusted to provide an optimum prophylactic or therapeutic response. A therapeutically effective amount is also one in which any toxic or detrimental side effects of the biologically active agent are outweighed in clinical terms by therapeutically beneficial effects. A non-limiting range for a therapeutically effective amount within the methods and formulations of the invention is 0.7 μg/kg to about 25 μg/kg. To promote weight loss, an intranasal dose of is administered at dose high enough to promote satiety but low enough so as not to induce any unwanted side-effects such as nausea. A preferred intranasal dose of PYY₃₋₃₆ is about 1 μg-10 μg/kg weight of the patient, most preferably from about 1.5 μg/kg to about 6 μg/kg weight of the patient. In a standard dose a patient will receive 40 μg to 2000 μg, more preferably about between 50 μg to 600 μg, most preferably 100 μg to 400 μg. Alternatively, a non-limiting range for a therapeutically effective amount of a biologically active agent within the methods and formulations of the invention is between about 0.001 pmol to about 100 pmol per kg body weight, between about 0.01 pmol to about 10 pmol per kg body weight, between about 0.1 pmol to about 5 pmol per kg body weight, or between about 0.5 pmol to about 1.0 pmol per kg body weight. Dosages within this range can be achieved by single or multiple administrations, including, e.g., multiple administrations per day, daily or weekly administrations. Repeated intranasal dosing with the formulations of the invention, on a schedule ranging from about 0.1 to 24 hours between doses, preferably between 0.5 and 24.0 hours between doses, will maintain normalized, sustained therapeutic levels of Y2 receptor-binding peptide to maximize clinical benefits while minimizing the risks of excessive exposure and side effects. This dose can be administered several times a day to promote satiety, preferably one half hour before a meal or when hunger occurs. The goal is to mucosally deliver an amount of the Y2 receptor-binding peptide sufficient to raise the concentration of the Y2 receptor-binding peptide in the plasma of an individual to mimic the concentration that would normally occur postprandially, i.e., after the individual has finished eating.

Dosage of Y2 agonists such as PYY may be varied by the attending clinician or patient, if self administering an over the counter dosage form, to maintain a desired concentration at the target site.

In an alternative embodiment, the invention provides compositions and methods for intranasal delivery of Y2 receptor-binding peptide, in which the Y2 receptor-binding peptide compound(s) is/are repeatedly administered through an intranasal effective dosage regimen that involves multiple administrations of the Y2 receptor-binding peptide to the subject during a daily or weekly schedule to maintain a therapeutically effective elevated and lowered pulsatile level of Y2 receptor-binding peptide during an extended dosing period. The compositions and method provide Y2 receptor-binding peptide compound(s) that are self-administered by the subject in a nasal formulation between one and six times daily to maintain a therapeutically effective elevated and lowered pulsatile level of Y2 receptor-binding peptide during an 8 hour to 24 hour extended dosing period.

The instant invention also includes kits, packages and multicontainer units containing the above described pharmaceutical compositions, active ingredients, and/or means for administering the same for use in the prevention and treatment of diseases and other conditions in mammalian subjects. Briefly, these kits include a container or formulation that contains one or more Y2 receptor-binding peptide proteins, analogs or mimetics, and/or other biologically active agents in combination with mucosal delivery enhancing agents disclosed herein formulated in a pharmaceutical preparation for mucosal delivery.

The intranasal formulations of the present invention can be administered using any spray bottle or syringe. An example of a nasal spray bottle is the, “Nasal Spray Pump w/ Safety Clip, Pfeiffer SAP #60548, which delivers a dose of 0.1 mL per squirt and has a diptube length of 36.05 mm. It can be purchased from Pfeiffer of America of Princeton, N.J. Intranasal doses of a Y2 receptor-binding peptide such as PYY can range from 0.1 μg/kg to about 1500 μg/kg. When administered in as an intranasal spray, it is preferable that the particle size of the spray are between 10-100 μm (microns) in size, preferably 20-100 μ/m in size.

To promote weight loss, an intranasal dose of a Y2 receptor-binding peptide PYY is administered at dose high enough to promote satiety but low enough so as not to induce any unwanted side-effects such as nausea. A preferred intranasal dose of a Y2 receptor-binding peptide such as PYY(3-36) is about 3 μg-10 μg/kg weight of the patient, most preferably about 6 μg/kg weight of the patient. In a standard dose a patient will receive 50 μg to 800 μg, more preferably about between 100 μg to 400 μg, most preferably 150 μg to about 200 μg. The a Y2 receptor-binding peptide such as PYY(3-36) is preferably administered at least ten minutes to one hour prior to eating to prevent nausea but no more than about twelve to twenty-four hours prior to eating. The patient is dosed at least once a day preferably before every meal until the patient has lost a desired amount of weight. The patient then receives maintenance doses at least once a week preferably daily to maintain the weight loss.”

As is shown by the data from the following examples, when administered intranasally to humans using the Y2 receptor-binding peptide formulation of the present invention, PYY(3-36) was found to reduce appetite. The examples also show that for the first time post-prandial physiological levels of a PYY peptide could be reached through an intranasal route of administration using the Y2 receptor-binding peptide formulations of the present invention in which PYY(3-36) was the Y2 receptor-binding peptide.

### Aerosal Nasal Administration of PYY

We have discovered that PYY in the formulations described above can be administered intranasally using a nasal spray or aerosol. This is surprising because many proteins and peptides have been shown to be sheared or denatured due to the mechanical forces generated by the actuator in producing the spray or aerosol. In this area the following definitions are useful.

Aerosol—A product that is packaged under pressure and contains therapeutically active ingredients that are released upon activation of an appropriate valve system.

Metered aerosol—A pressurized dosage form comprised of metered dose valves, which allow for the delivery of a uniform quantity of spray upon each activation.

Powder aerosol—A product that is packaged under pressure and contains therapeutically active ingredients in the form of a powder, which are released upon activation of an appropriate valve system.

Spray aerosol—An aerosol product that utilizes a compressed gas as the propellant to provide the force necessary to expel the product as a wet spray; it generally applicable to solutions of medicinal agents in aqueous solvents.

Spray—A liquid minutely divided as by a jet of air or steam. Nasal spray drug products contain therapeutically active ingredients dissolved or suspended in solutions or mixtures of excipients in nonpressurized dispensers.

Metered spray—A non-pressurized dosage form consisting of valves that allow the dispensing of a specified quantity of spray upon each activation.

Suspension spray—A liquid preparation containing solid particles dispersed in a liquid vehicle and in the form of course droplets or as finely divided solids.

The fluid dynamic characterization of the aerosol spray emitted by metered nasal spray pumps as a drug delivery device (“DDD”). Spray characterization is an integral part of the regulatory submissions necessary for Food and Drug Administration (“FDA”) approval of research and development, quality assurance and stability testing procedures for new and existing nasal spray pumps.

Thorough characterization of the spray's geometry has been found to be the best indicator of the overall performance of nasal spray pumps. In particular, measurements of the spray's divergence angle (plume geometry) as it exits the device; the spray's cross-sectional ellipticity, uniformity and particle/droplet distribution (spray pattern); and the time evolution of the developing spray have been found to be the most representative performance quantities in the characterization of a nasal spray pump. During quality assurance and stability testing, plume geometry and spray pattern measurements are key identifiers for verifying consistency and conformity with the approved data criteria for the nasal spray pumps. The following definitions apply to the properties of the spray.

Plume Height—the measurement from the actuator tip to the point at which the plume angle becomes non-linear because of the breakdown of linear flow. Based on a visual examination of digital images, and to establish a measurement point for width that is consistent with the farthest measurement point of spray pattern, a height of 30 mm is defined for this study

Major Axis—the largest chord that can be drawn within the fitted spray pattern that crosses the COMw in base units (mm)

Minor Axis—the smallest chord that can be drawn within the fitted spray pattern that crosses the COMw in base units (mm)

Ellipticity Ratio—the ratio of the major axis to the minor axis

D₁₀—the diameter of droplet for which 10% of the total liquid volume of sample consists of droplets of a smaller diameter (μm)

D₅₀—the diameter of droplet for which 50% of the total liquid volume of sample consists of droplets of a smaller diameter (μm), also known as the mass median diameter

D₉₀—the diameter of droplet for which 90% of the total liquid volume of sample consists of droplets of a smaller diameter (μm)

Span—measurement of the width of the distribution, The smaller the value, the narrower the distribution. Span is calculated as:

$$\frac{( {D}_{90} - {D}_{10} )}{{D}_{50}} .$$

% RSD—percent relative standard deviation, the standard deviation divided by the mean of the series and multiplied by 100, also known as % CV.

A nasal spray device is comprised of a bottle into which the PYY formulation is placed, and an actuator, which when actuated or engaged forces a spray plume, of PYY out of the spray bottle through the actuator. The bottles may be smooth glass bottles comprised of Type I borosilicate glass. The bottles may have a screw top and a concave bottom. The caps may be trifoil-Lined polypropylene. The Tri-Foil WP consists of a 0.0005″ clear polyester that is bonded by 0.00067″ white LDPE to a 0.0035″ aluminum foil then bonded to a LDPE film/foam/film co-extrusion. All components of this liner are GRAS. The caps may be comprised of polypropylene and are appropriately threaded for use with the intended vials.

### EXAMPLES

### Example 1

### In Vitro Tissue Model Evaluation of Various PYY3-36 Formulations

In vitro permeation of PYY₃₋₃₆ in the presence of various excipients (EDTA, polysorbate 80 (Tween 80), oleic acid, sorbitol, and ethanol) was evaluated. The formulation was adjusted to pH 4 with 10 mM citrate buffer (citric acid/sodium citrate). Various formulations tested are presented in Table 1. All samples were clear and colorless.

TABLE 1
Description of Formulations Tested in Example 1

| | | | Conc. (%) | | | |
|---|---|---|---|---|---|---|
| Sample | Conc. (mg/ml) | | | Tween | Oleic | Sorbitol |
| # | PYY | EDTA | Ethanol | 80 | acid | (mM) |
| 1 | 2 | 0 | 0 | 0 | 0 | 0 |
| | | | | | | (140 mM NaCl) |
| 2 | 2 | 1 | 0 | 0 | 0 | 195 |
| 3 | 2 | 10 | 0 | 0 | 0 | 135 |
| 4 | 2 | 0 | 1 | 0 | 0 | 0 |
| 5 | 2 | 0 | 2 | 0 | 0 | 0 |
| 6 | 2 | 0 | 0 | 0.1 | 0 | 200 |
| 7 | 2 | 0 | 0 | 1 | 0 | 200 |
| 8 | 2 | 0 | 0 | 10 | 0 | 170 |
| 9 | 2 | 0 | 0 | 10 | 0.1 | 170 |
| 10 | 2 | 0 | 0 | 10 | 0.5 | 170 |
| 11 | 2 | 1 | 1 | 0 | 0 | 0 |
| 12 | 2 | 10 | 2 | 0 | 0 | 0 |
| 13 | 2 | 1 | 0 | 0.1 | 0 | 190 |
| 14 | 2 | 10 | 0 | 10 | 0 | 105 |
| 15 | 2 | 1 | 0 | 10 | 0.1 | 165 |
| 16 | 2 | 0 | 1 | 10 | 0.1 | 0 |
| 17 | 2 | 10 | 0 | 10 | 0.5 | 105 |
| 18 | 2 | 0 | 2 | 10 | 0.5 | 0 |
| 19 | 2 | 1 | 1 | 10 | 0.1 | 0 |
| 20 | 2 | 10 | 2 | 10 | 0.5 | 0 |

| | |
|---|---|
| 21 | Medium |
| 22 | Triton X |

Samples were evaluated in an in vitro tissue model. The cell line used was normal, human-derived tracheal/bronchial epithelial cells (EpiAirway™ Tissue Model, MatTek Corporation). The cells were provided as inserts grown to confluency on Millipore Millicell-CM filters comprised of transparent hydrophilic Teflon (PTFE). Upon receipt, the membranes were cultured in 1 ml basal media (phenol red-free and hydrocortisone-free Dulbecco's Modified Eagle's Medium (DMEM)) at 37° C./5% CO₂ for 24-48 hours before use. Inserts were feed for each day of recovery.

Each tissue insert was placed in an individual well containing 1 ml of MatTek basal media. On the apical surface of the inserts, 50 μl of test formulation was applied according to study design in Table 1, and the samples were placed on a shaker (˜100 rpm) for 1 h at 37° C. The underlying culture media samples were stored at 4° C. for up to 48 hours for lactate dehydrogenase (LDH, cytotoxicity) and sample permeation (enzyme immunoassay (EIA)) evaluations. Transepithelial electrical resistance (TER) was measured before and after a 1-h incubation. Following the incubation, the cell inserts were analyzed for cell viability via the mitochondrial dehydrogenase (MDH) assay.

1. Electrical Resistance Across a Monocellular Layer

TER measurements were accomplished using the Endohm-12 Tissue Resistance Measurement Chamber connected to the EVOM Epithelial Voltohmmeter (World Precision Instruments, Sarasota, Fla.) with the electrode leads. The electrodes and a tissue culture blank insert were equilibrated for at least 20 minutes in MatTek medium with the power off prior to check calibration. The background resistance was measured with 1.5 ml Media in the Endohm tissue chamber and 300 μl Media in the blank insert. The top electrode was adjusted so that it was close to, but not making contact with, the top surface of the insert membrane. Background resistance of the blank insert was about 5-20 ohms. For each TER determination, 300 μl of MatTek medium was added to the insert followed by placement in the Endom chamber. Resistance was expressed as (resistance measured−blank)×0.6 cm².

The results show that the negative control and media control did not exhibit any significant change in TER after one hour exposure. The Triton control shows essentially a complete reduction in TER. Samples with EDTA and ethanol exhibited a decrease in TER, consistent with opening of tight junctions.

2. Cell Viability

Cell viability was assessed using the MTT assay (MTT-100, MatTek kit). Thawed and diluted MTT concentrate was pipetted (300 μl) into a 24-well plate. Tissue inserts were gently dried, placed into the plate wells, and incubated at 37° C. for 3 hours. After incubation, each insert was removed from the plate, blotted gently, and placed into a 24-well extraction plate. The cell culture inserts were then immersed in 2.0 ml of the extractant solution per well (to completely cover the sample). The extraction plate was covered and sealed to reduce evaporation of extractant. After an overnight incubation at room temperature in the dark, the liquid within each insert was decanted back into the well from which it was taken, and the inserts discarded. The extractant solution (200 μl in at least duplicate) was pipetted into a 96-well microtiter plate, along with extract blanks. The optical density of the samples was measured at 550 nm on a plate reader.

All samples and the negative and media controls exhibited an acceptable cell viability, i.e., at least 80% compared to the media control. The Triton X control substantially decreased cellular viability, as expected.

3. Cytoxicity

The amount of cell death (cytotoxicity) was assayed by measuring the loss of lactate dehydrogenase (LDH) from the cells using a CytoTox 96 Cytoxicity Assay Kit (Promega Corp., Madison, Wis.).

LDH analysis of the apical media was evaluated. The appropriate amount of media was added to the apical surface in order to total 300 uL, taking into consideration the initial sample loading volume. The inserts were shaken for 5 minutes, and then 150 uL of the apical media was removed and dispensed into eppendorf tubes and centrifuged at 10000 rpm for 3 minutes. A volume of 2 uL of the supernatant was removed and added to a 96 well plate. A volume of 48 uL of media was used to dilute the supernatant to make a 25× dilution. For LDH analysis of the basolateral media, 50 uL of sample was loaded into a 96-well assay plate. Fresh, cell-free culture medium was used as a blank. Fifty microliters of substrate solution was added to each well and the plates incubated for 30 minutes at room temperature in the dark. Following incubation, 50 μl of stop solution was added to each well and the plates read on an optical density plate reader at 490 nm. All samples and the negative and media controls had relatively low cytotoxicity by basolateral LDH assay, i.e., no more than 20% compared to the media control. The Triton X control had relative high cytotoxicity, as expected.

4. Cellular Permeation

PYY₃₋₃₆ EIA kits were purchased from Phoenix Pharmaceuticals, Inc. (Belmont, Calif.), and the assay was conducted following the provided instructions.

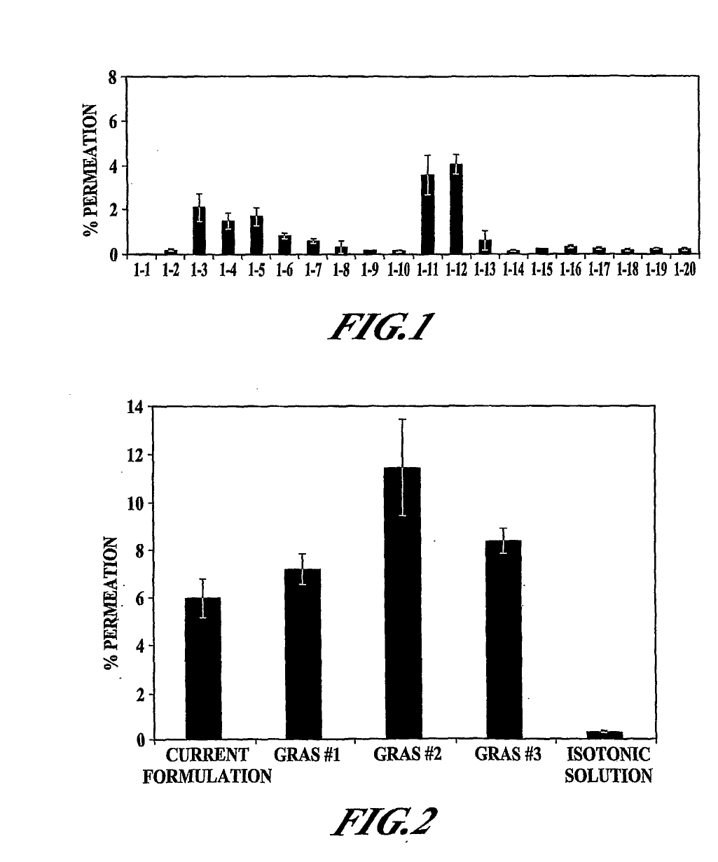

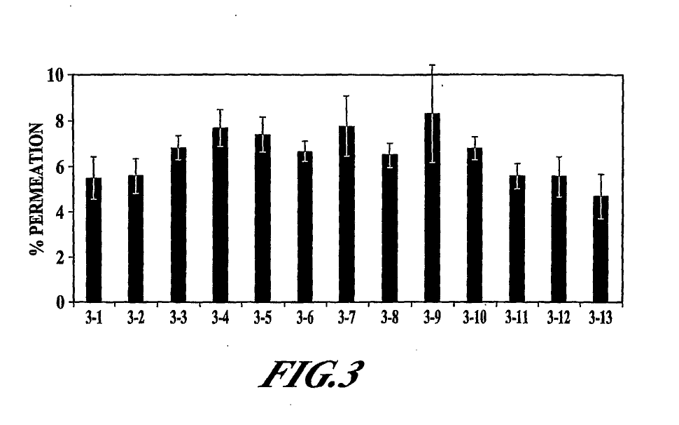

Permeation results (FIG. 1) showed 10 mM EDTA or 1-2% ethanol provided substantial % permeation, i.e., about 1.5 to 2% drug permeated in one hour. In contrast, very low % permeation, e.g., <0.3% was observed for the negative control (isotonic, no permeation enhancers). The greatest % permeation was observed for samples 11 (1 mg/mL EDTA, 1% ethanol) and 12 (10 mg/mL EDTA, 2% ethanol). The combination of EDTA and ethanol provide for reduction in TER, consistent with opening of tight junctions and increase PYY3-36 permeation, with acceptable low cytotoxicity and high cell viability.

### Example 2

### In Vitro Evaluation of Various PYY3-36 Formulations

The objective was to further examine the effect of ethanol, EDTA, and Tween 80 as permeation enhancers for PYY₃₋₃₆. Formulations were adjusted to pH 4 with 10 mM citrate buffer (citric acid/sodium citrate). The various formulations tested in Example 2 are presented in Table 2. In addition to these samples, a negative isotonic control, a cell culture media control, and a Triton-X control were also included.

TABLE 2
Description of Formulations Tested in Example 2

| Formulation | Description |
|---|---|
| Current | 45 mg/mL MβCD, 1 mg/mL DDPC, 1 mg/mL EDTA, |
| | 10 mM citrate buffer (pH 5.0), 25 mM lactose, |
| | 100 mM sorbitol, 0.5% CB |
| GRAS #1 | 1 mg/mL EDTA, 1% EtOH, 10 mM acetate buffer |
| | (pH 4.0), 0.02% BZK |
| GRAS #2 | 10 mg/mL EDTA, 2% EtOH, 10 mM acetate buffer |
| | (pH 4.0), 0.02% BZK |
| GRAS #3 | 10 mg/mL EDTA, 10 mM acetate buffer (pH 4.0), |
| | 0.02% BZK |
| Saline | Isotonic saline |

The various samples in Table 2 were examined for TER, MTT, LDH and PYY₃₋₃₆ permeation; the methodologies for the various assays were accomplished as described in Example 1.

The negative control and media control did not exhibit any significant change in TER after one hour exposure. The Triton control showed essentially a complete reduction in TER. All test samples revealed a drop in TER. These data illustrate that EDTA, ethanol, Tween 80, and combinations of these excipients decrease TER, which is consistent with opening of tight junctions.

All samples and the negative and media controls had relatively low cytotoxicity by the basolateral LDH assay, i.e., no more than 20% toxicity compared to the media control. The Triton X control had relative high cytotoxicity.

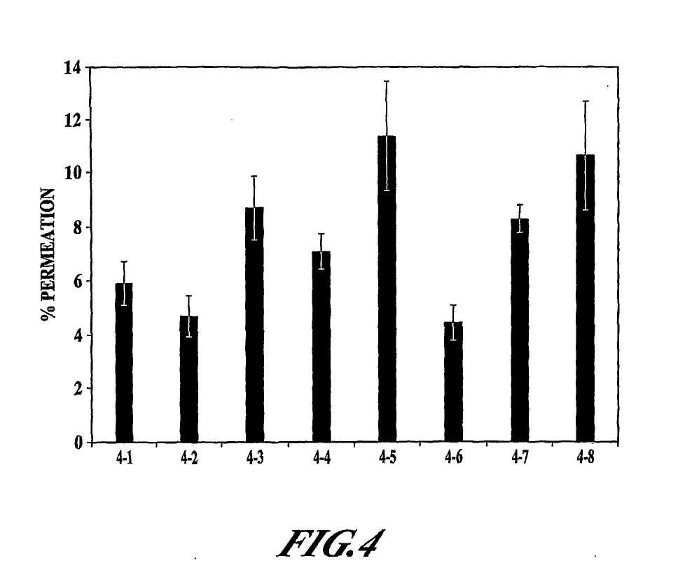

Data for % permeation are shown in FIG. 2. The negative control had very low permeation, consistent with Example 1. The three GRAS samples exhibited substantially high permeation under the conditions tested. These data further confirm that EDTA, ethanol, and Tween 80, and combinations of these excipients reduce TER and enhanced PYY3-36 permeation, with acceptable low cytotoxicity.

### Example 3

### In Vitro Tissue Model Evaluation of Various PYY3-36 Formulations

The objective of this study was to further examine the effect of ethanol, EDTA, and Tween 80 as potential permeation enhancers for PYY₃₋₃₆.

The in vitro permeation of PYY₃₋₃₆ in the presence of various excipients (EDTA, ethanol, Tween 80, DDPC, and methyl-beta-cyclodextrin) was evaluated. Formulations were adjusted to pH 4.2-4.3 with 10 mM citrate buffer (citric acid/sodium citrate). The various formulations tested are shown in Table 3. In addition to these samples, a negative isotonic control, a cell culture media control, and a Triton X control were also included. Sample 3-1 contained a combination of methyl-beta-cyclodextrin (M-β-CD), DDPC, and EDTA, in a combination shown previously to provide enhancement of PYY3-36 permeation (U.S. patent application Ser. No. 10/768,288 [Publication No. 20040209807]).

The various samples in Table 3 were examined for TER, MTT, LDH, and PYY₃₋₃₆ permeation; the methodologies for the various assays were accomplished as described in Example 1.

TABLE 3
Description of Formulations Tested in Example 3

| | Conc. (mg/ml) | | | | | |
|---|---|---|---|---|---|---|
| | | | | | | Tween |
| Sample # | PYY | M-β-CD | DDPC | EDTA | EtOH | 80 |
| 3-1 | 2 | 45 | 1 | 1 | 0 | 0 |
| 3-2 | 2 | 0 | 0 | 1 | 10 | 1 |
| 3-3 | 2 | 0 | 0 | 10 | 20 | 1 |
| 3-4 | 2 | 0 | 1 | 10 | 20 | 0 |
| 3-5 | 2 | 0 | 0 | 5 | 10 | 0 |
| 3-6 | 2 | 0 | 0 | 5 | 20 | 0 |
| 3-7 | 2 | 0 | 0 | 5 | 30 | 0 |
| 3-8 | 2 | 0 | 0 | 10 | 10 | 0 |
| 3-9 | 2 | 0 | 0 | 10 | 20 | 0 |
| 3-10 | 2 | 0 | 0 | 10 | 30 | 0 |
| 3-11 | 2 | 0 | 0 | 20 | 10 | 0 |
| 3-12 | 2 | 0 | 0 | 20 | 20 | 0 |
| 3-13 | 2 | 0 | 0 | 20 | 30 | 0 |

All samples were clear and colorless. The media and negative controls did not exhibit any significant change in TER after one hour exposure. Samples 3-1 through 3-13 all exhibited a substantial decrease in TER in contrast to the media control, showing EDTA, ethanol, Tween 80 and combinations of these excipients decreased TER. The data further show that a formulation containing methyl-beta-cyclodetrin, EDTA and DDPC decreased TER.

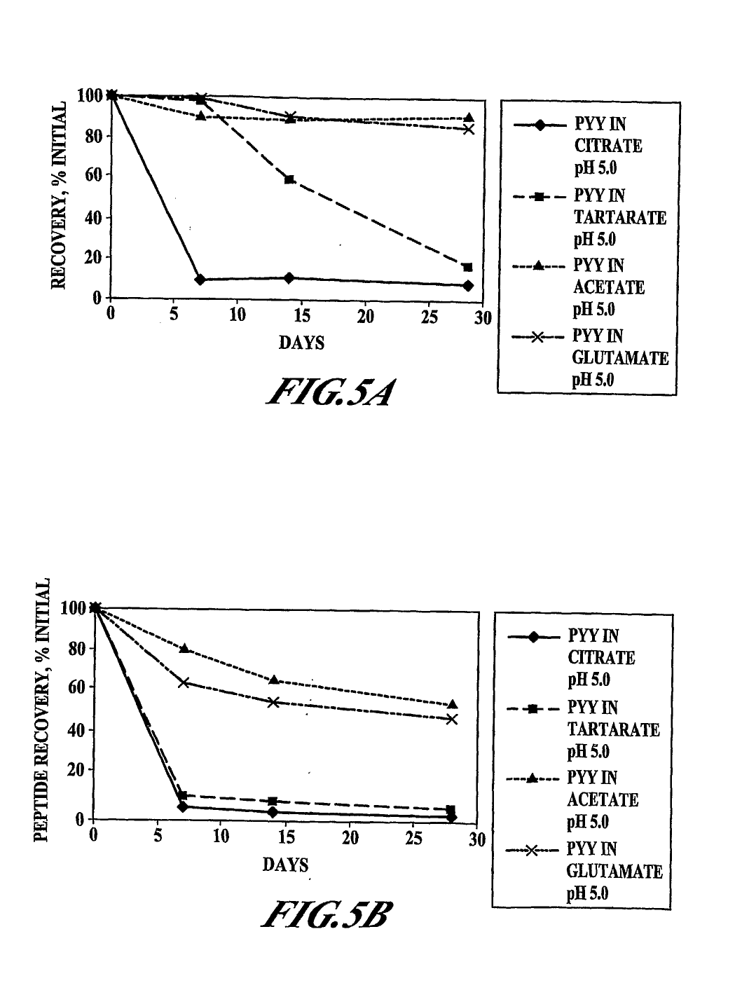

Data for % permeation (FIG. 3) show that relatively high % permeation was exhibited for all samples 3-1 through 3-13. The negative control had very low % permeation, consistent with Example 1. These data further confirm that EDTA, ethanol, Tween 80, and combinations thereof provide for reduction in TER, consistent with opening of tight junctions and increased PYY₃₋₃₆ permeation. These formulations achieve permeation comparable or better than that for previously described formulations containing EDTA, DDPC, and methyl-beta-cyclodextrin (U.S. patent application Ser. No. 10/768,288).

### Example 4

### In Vitro Tissue Model Evaluation of Various PYY3-36 Formulations

The objective of this study was to further examine the effect of ethanol, EDTA, and Tween 80 as potential permeation enhancers for PYY₃₋₃₆. In this example, different buffers were tested (citrate buffer, acetate buffer, and glutamate buffer), as well as different preservative (chlorobutanol and benzalkonium chloride). The data for all formulations were compared to a formulation with methyl-beta-cyclodextrin, DDPC, and EDTA.

The in vitro permeation of PYY₃₋₃₆ in the presence of various excipients (EDTA, ethanol, Tween 80, DDPC, and methyl-beta-cyclodextrin) was evaluated. Formulations were adjusted to pH 4 with 10 mM citrate buffer (citric acid/sodium citrate). The various formulations tested are presented in Table 4. In addition to these samples, a negative isotonic control, a cell culture media control, and a Triton X control were also included. Sample 4-1 contained a formulation with methyl-beta-cyclodextrin, DDPC, and EDTA, previously shown to provide enhancement of PYY₃₋₃₆ permeation (U.S. patent application Ser. No. 10/768,288).

TABLE 4
Description of Formulations Tested in Example 4

| Sample | Concentration (mg/mL) | | | | | | pH, buffer, and |
|---|---|---|---|---|---|---|---|
| # | PYY | Me-b-CD | DDPC | EDTA | EtOH | Tween 80 | preservative |
| 4-1 | 6 | 45 | 1 | 1 | 0 | 0 | 5, citrate, CB |
| 4-2 | 6 | 0 | 0 | 1 | 10 | 1 | 4.3, acetate, BZK |
| 4-3 | 6 | 0 | 0 | 10 | 20 | 1 | 4.3, acetate, BZK |
| 4-4 | 6 | 0 | 0 | 1 | 10 | 0 | 4.3, acetate, BZK |
| 4-5 | 6 | 0 | 0 | 10 | 20 | 0 | 4.3, acetate, BZK |
| 4-6 | 6 | 0 | 0 | 10 | 0 | 0 | 4.3, acetate, BZK |
| 4-7 | 6 | 0 | 0 | 10 | 0 | 0 | 4.3, acetate, BZK |
| 4-8 | 6 | 0 | 0 | 10 | 20 | 0 | 4.3, glutamate, BZK |

The various samples in Table 4 were examined for TER, MTT, LDH, and PYY₃₋₃₆ permeation; the methodologies for the various assays were accomplished as described in Example 1. In Table 4, “BZK”=0.2 mg/mL benzalkonium chloride and “CB”=5 mg/mL chlorobutanol.

The Triton X control showed essentially a complete reduction in TER. The negative and media control showed no change in TER. All test samples exhibited a substantial decrease in TER in contrast to the negative control. The data show that acetate and glutamate buffers can be substituted for citrate buffer in the PYY₃₋₃₆ formulation. The data further support that preservatives such as benzalkonium chloride and chlorobutanol can be added to the PYY₃₋₃₆ formulation.

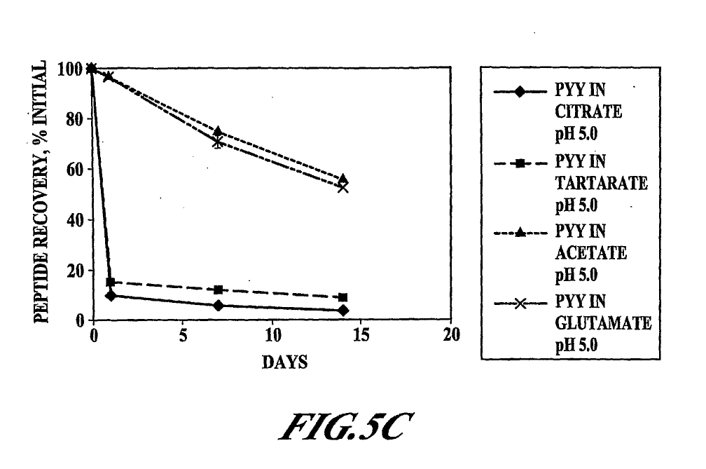

Permeation data (FIG. 4) show relatively high % permeation for all samples, and the highest % permeation was exhibited for 4-4, 4-6, and 4-9. These data further confirm the permeation enhancing effect of EDTA, ethanol, Tween 80, and combinations thereof. Acetate and glutamate buffers were substituted for citrate buffer without loss of permeation. Further, benzalkonium chloride and chlorobutanol were successfully added as preservatives.

These data confirm that EDTA, ethanol, and Tween 80 formulations can achieve % permeation comparable or better than that for the previously described formulation containing EDTA, DDPC, and methyl-beta-cyclodextrin; acetate and glutamate buffers can be substituted for citrate buffer in the PYY₃₋₃₆ formulation; and preservatives such as benzalkonium chloride and chlorobutanol can be added to the PYY₃₋₃₆ formulation.

### Example 5

### Pharmacokinetic Testing of Intranasal PYY3-36 Formulations Containing Acetate Buffer, Ethanol, and EDTA

Pharmacokinetic testing of PYY₃₋₃₆ in various intranasal formulations was tested in mammals. The formulations included EDTA and ethanol as permeation enhancers (acetate buffer; pH 4.0). For comparison, a formulation was also dosed intranasally containing methyl-beta-cyclodextrin, DDPC, and EDTA as permeation enhancers (this combination of excipients was shown previously to provide enhancement of PYY₃₋₃₆ permeation (U.S. patent application Ser. No. 10/768,288). Further, a formulation devoid of enhancers was dosed in order to elucidate the potency of the permeation enhancers to improve drug delivery.

The various formulations tested in Example 5 are described in Table 5. Sample 5-1 contained methyl-beta-cyclodextrin, DDPC, and EDTA as enhancers and CB as a preservative, 5-1 was dosed IN for comparison. Samples 5-2, 5-3 and 5-4 contained EDTA at either 1 or 10 mg/mL and ethanol at either 0, 10 or 20 mg/mL, and BZK as a preservative. Sample 5-5 was formulated in buffer and was devoid of permeation enhancers.

TABLE 5
Description of Formulations Tested in Example 5

| | | PYY₃₋₃₆ | |
|---|---|---|---|
| Sample | Dose | concentration | |
| # | (μg/kg) | (mg/mL) | Formulation |
| 5-1 | 205 | 13.6 | 45 mg/mL MβCD, 1 mg/mL DDPC, |
| | | | 1 mg/mL EDTA, 10 mM citrate, pH |
| | | | 5.0, 25 mM lactose, 100 mM |
| | | | sorbitol, 0.5% CB |
| 5-2 | 205 | 13.6 | 1 mg/mL EDTA, 1% EtOH, 10 mM |
| | | | acetate, pH 4.0, 0.02% BZK |
| 5-3 | 205 | 13.6 | 10 mg/mL EDTA, 2% EtOH, 10 mM |
| | | | acetate, pH 4.0, 0.02% BZK |
| 5-4 | 205 | 13.6 | 10 mg/mL EDTA, 10 mM acetate |
| | | | (pH 4.0), 0.02% BZK |
| 5-5 | 205 | 13.6 | Isotonic saline |

Pharmacokinetic (PK) evaluation in New Zealand white rabbits adhered to the Principles of Laboratory Animal Care (NIH publication 86-23, revised 1985). Blood samples were taken from the marginal ear vein at pre-dose and 2.5, 5, 10, 15, 30, 45, 60, and 120 min after IN dosing. The concentration of PYY₃₋₃₆ in plasma was determined by EIA (Foerder C., et al., Quantitative Determination of Peptide YY3-36 in Plasma by Radioimmunoassay, AAPS 2004 National Biotechnology Conference, Boston, Mass., May 2004). PK calculations were performed using WinNonlin software (Pharsight Corporation, Version 4.0, Mountain View, Calif.) employing a non-compartmental model approach. Data are presented as mean±standard error.

The PK results are summarized in Table 6. The Tₘₐₓ by IN route was between about 26-43 for all samples. Sample 5-1 had the highest Cₘₐₓ and AUC, the latter was 27-fold improved compared to the case of no enhancers (sample 5-5). Samples 5-2 and 5-3 exhibited a lower standard deviation in their Cₘₐₓ compared to sample 5-1. Also, sample 5-2 exhibited a lower standard deviation in Aucₗₐₛₜ compared to sample 5-1. Sample 5-2 exhibited nearly the same Aucₗₐₛₜ as sample 5-1.

TABLE 6
PK Summary of Formulations Tested in Example 5

| | | Cmax | Cmax | Auclast | | Fold |
|---|---|---|---|---|---|---|
| | Tmax | (pg/ | SD | (min*pg/ | AUC SD | improvement |
| Sample | (min) | mL) | (%) | mL) | (%) | in AUC * |
| 5-1 | 29 | 20713 | 46 | 1002914 | 43 | 27 |
| 5-2 | 38 | 14271 | 23 | 929365 | 31 | 25 |
| 5-3 | 43 | 13954 | 22 | 783633 | 44 | 21 |
| 5-4 | 40 | 8902 | 75 | 722448 | 79 | 20 |
| 5-5 | 26 | 517 | n/d | 36476 | n/d | 1 |

n/d = not determined;
* compared to no enhancers (8-5)

All groups containing the EDTA and/or ethanol combinations (samples 5-2, 5-3 and 5-4) showed substantially improved permeation compared to no enhancers, up to about 20- to 25-fold greater.

### Example 6

### Effect of Various Buffers on Thermal Stress Stability for PYY3-36 in the Absence of Any Additional Excipients

Formulations were manufactured as outlined in Table 7. The buffers tested included citrate, tartarate, acetate, and glutamate. In all cases, PYY₃₋₃₆ was present at 1 mg/mL and the pH was 5.0. 1-cc amber non-silanized vials were filled with the test formulations, 1 mL fill per vial, and the vials were fitted with a trifoil-lined cap. The vials were purchased from SGD Glass Inc. (New York, N.Y.). These vials had a screw top and a concave bottom (U-shape configuration). The vials were comprised of Type I borosilicate glass. The caps were purchased from O'Berk Company (Union, N.J.) and were comprised of polypropylene and were appropriately threaded for use with the intended vials. The caps were Tri-Foil lined. The Tri-Foil WP consisted of a 0.0005″ clear polyester that was bonded by 0.00067″ white LDPE to a 0.0035″ aluminum foil then bonded to a LDPE film/foam/film co-extrusion. All components of the liner were GRAS (Generally Recognized as Safe).

Vials were stored at 25, 40 and 50° C., and at various time points were tested by HPLC to examine chemical stability, as reflected in peptide recovery (% peptide measured relative to the data at t=0). The HPLC method uses a 5-micron C18 column (Supelco, BIO Wide-pore, 250×4.6 mm) at 45° C. with mobile phase components of 0.1% trifluoroacetic acid (A) and 0.08% trifluoroacetic acid in acentonitrile (B) delivered isocratically at 27% A/73% B. Detection was by UV at 210 nm. Quantitation was carried out by external standard method.

TABLE 7
Formulations Evaluated in Example 6

| | | Citrate | Tartarate | Acetate | Glutamate | |
|---|---|---|---|---|---|---|
| | PYY₃₋₃₆ | Buffer | Buffer | Buffer | Buffer | |
| Group # | (mg/mL) | (mM) | (mM) | (mM) | (mM) | pH |
| 1-1 | 1 | 10 | — | — | — | 5.0 |
| 1-2 | 1 | — | 10 | — | — | 5.0 |
| 1-3 | 1 | — | — | 10 | — | 5.0 |
| 1-4 | 1 | — | — | — | 10 | 5.0 |

The HPLC data show that the best stability (highest recovery after storage at the various conditions) was achieved using the acetate and glutamate buffers. The peptide recovery results are shown in FIGS. 5A, 5B and 5C for 25, 40 and 50° C., respectively.

The results of this experiment are illuminating in that those buffers that best preserved PYY₃₋₃₆ stability were monovalent buffers, whereas those that did not improve PYY₃₋₃₆ stability were polyvalent buffers. Monovalent buffers likely increase PYY₃₋₃₆ stability under thermal stress.

### Example 7

### Effect of Buffer Type and pH on Thermal Stress Stability for PYY3-36 in the Presence of Sorbitol as Tonicifier

Formulations were manufactured as outlined in Table 8. The buffers tested were citrate, acetate, and glutamate. In all cases, PYY3-36 was present at 2 mg/mL and sorbitol was present to provide osmolarity of 225 mOsm. The pH was varied from 3.5 to 5.0. These formulations were then filled into 1-cc amber non-silanized vials, 1 mL fill per vial, and fitted with a trifoil-lined polypropylene cap. Samples were stored and tested for recovery as described in Example 6.

TABLE 8
Formulations Evaluated in Example 7

| | | Citrate | Acetate | Glutamate | | |
|---|---|---|---|---|---|---|
| | PYY | Buffer | Buffer | Buffer | Sorbitol | |
| Group # | (mg/mL) | (mM) | (mM) | (mM) | (mM) | pH |
| 2-1 | 2 | 10 | 0 | 0 | 210 | 5.0 |
| 2-2 | 2 | 10 | 0 | 0 | 210 | 4.5 |
| 2-3 | 2 | 10 | 0 | 0 | 210 | 4.0 |
| 2-4 | 2 | 10 | 0 | 0 | 210 | 3.5 |
| 2-5 | 2 | 0 | 10 | 0 | 210 | 5.0 |
| 2-6 | 2 | 0 | 10 | 0 | 210 | 4.5 |
| 2-7 | 2 | 0 | 10 | 0 | 210 | 4.0 |
| 2-8 | 2 | 0 | 10 | 0 | 210 | 3.5 |
| 2-9 | 2 | 0 | 0 | 10 | 210 | 5.0 |
| 2-10 | 2 | 0 | 0 | 10 | 210 | 4.5 |
| 2-11 | 2 | 0 | 0 | 10 | 210 | 4.0 |
| 2-12 | 2 | 0 | 0 | 10 | 210 | 3.5 |
| 2-13 | 2 | 0 | 0 | 0 | 0 | 5.0 |
| 2-14 | 2 | 0 | 0 | 0 | 0 | 4.5 |
| 2-15 | 2 | 0 | 0 | 0 | 0 | 4.0 |
| 2-16 | 2 | 0 | 0 | 0 | 0 | 3.5 |

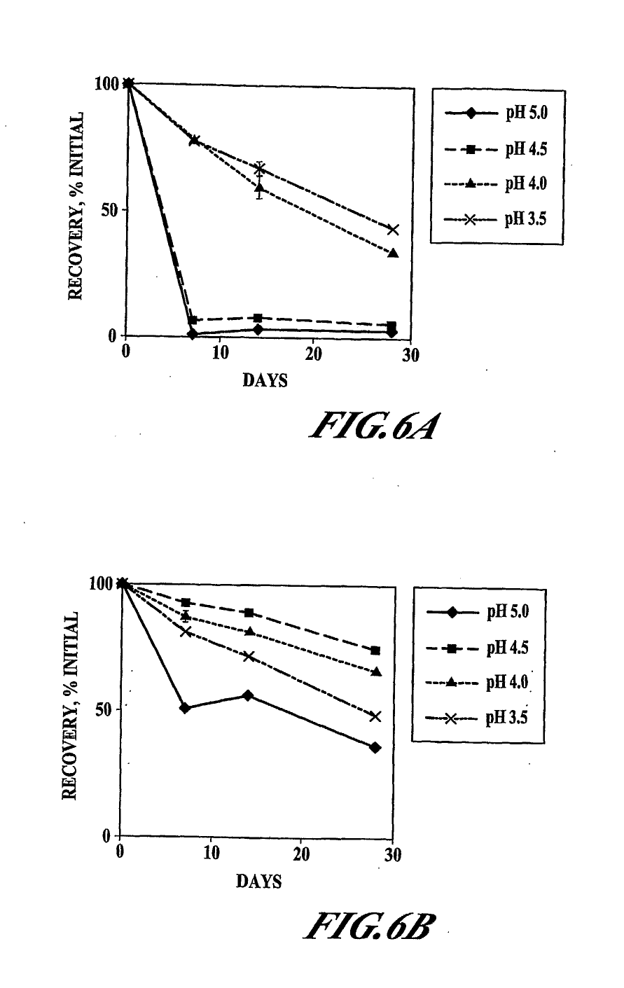

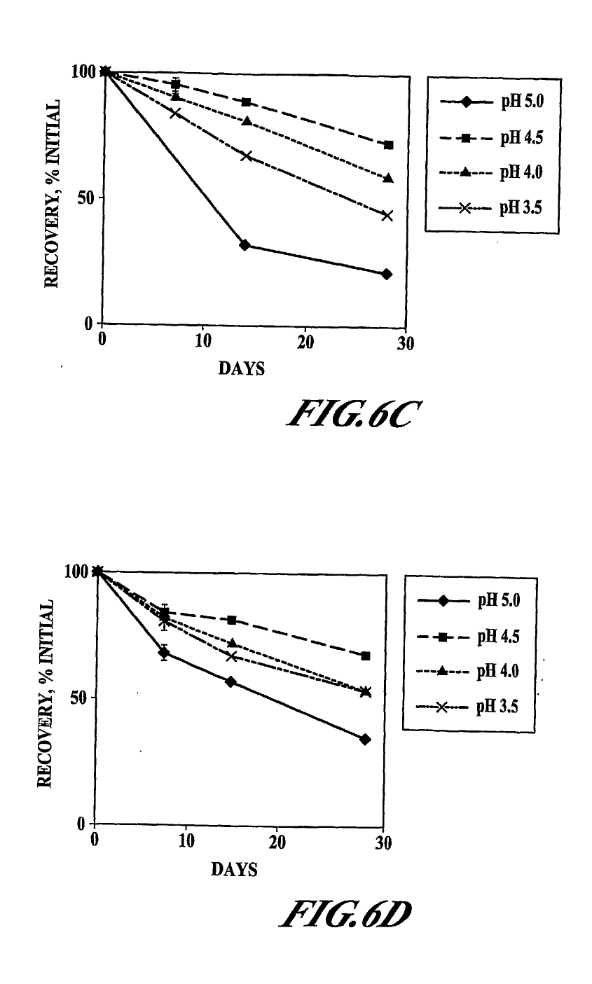

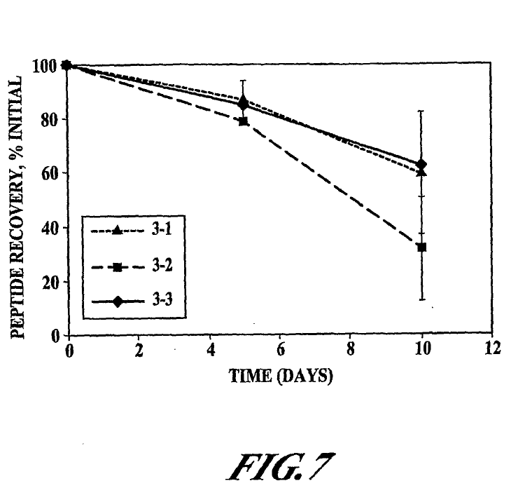

The HPLC data show that the best performing formulations for thermal stability over the temperatures evaluated are the acetate and glutamate buffers as well as the unbuffered formulations. The peptide recovery results are depicted in FIGS. 6A, 6B, 6C and 6D for cases where the buffer was citrate, acetate, glutamate or no buffer, respectively.

As in Example 6, the best-performing formulations are those that contain either a monovalent buffer (i.e., acetate or glutamate) or that do not contain a buffer over the pH range and temperatures evaluated. Those formulations containing a polyvalent buffer (i.e., citrate) did not reach optimal performance. In addition, it appears that optimal stability-maintaining pH for PYY₃₋₃₆ appears to be pH 3.5-pH 4.5 regardless of buffer used.

### Example 8

### Thermal Stability and Atomization Stress Stability for Various PYY3-36 Formulations

The objective of this study was to examine stability against thermal and atomization stresses for PYY₃₋₃₆ formulations containing ethanol, EDTA, and Tween 80 as potential permeation enhancers for PYY₃₋₃₆. In this example, different buffers were added (acetate buffer and glutamate buffer), as well a preservative to allow for multi-use formulations (benzalkonium chloride).

Solutions were filled at ˜3.9 mL in 3 mL non-silanized amber glass vials. Vials were affixed with 100 μL actuators. The actuators were purchased from Pfeiffer of America (Princeton, N.J.). Vials were primed by actuating until the first full spray was achieved. After the first complete spray was observed, an additional two actuations were performed to confirm full priming. After priming was complete, the next spray was considered the first spray of the study. All actuations were conducted by hand.

Vials were stored at 30° C./65% relative humidity between all sprays (during the day as well as overnight). After removal from the chamber, vials were sprayed (within 5 min.) and then returned to the chamber. There was a minimum of 1 hour between all sprays. Vials were sprayed three times per day (TID) for 10 days.

The HPLC method was conducted as described in Example 1. Data for peptide stability are presented as % recovery (concentration of native peptide relative to that initially at t=0) and % purity (peak area of native peptide divided by area of all peptide-associated peaks). HPLC data for peptide content was measured for day=0, day=5 and day=10 of the exposure to elevated temperature and atomization stress of thrice daily spraying.

The various formulations tested in Example 8 are described in Table 9. All samples contained 6 mg/mL PYY₃₋₃₆. Samples contained 10 mg/mL EDTA, 20 mg/mL ethanol, 0.02% benzalkonium chloride (BZK) (as a representative preservative to allow for multi-use), and either 0 or 1 mg/mL Tween 80. Samples 3-1 and 3-2 contained 10 mM acetate buffer (acetic acid/sodium acetate buffer system) at pH 4.3. Sample 3-3 contained 10 mM glutamate buffer (glutamic acid/sodium glutamate buffer system) at pH 4.3.

TABLE 9
Description of Formulations Tested in Example 8

| | | | Tween 80 | Buffer |
|---|---|---|---|---|
| Sample # | EDTA (mg/mL) | EtOH (mg/mL) | (mg/mL) | (10 mM) |
| 3-1 | 10 | 20 | 1 | Acetate |
| 3-2 | 10 | 20 | 0 | Acetate |
| 3-3 | 10 | 20 | 0 | Glutamate |

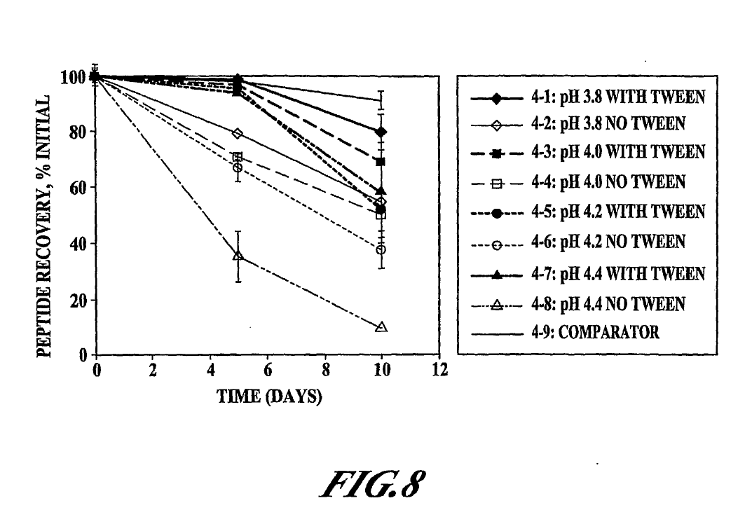

HPLC data for peptide content at day=0, day=5 and day=10 of the exposure to elevated temperature and atomization stress of thrice daily spraying are depicted in FIG. 7.

The data show that in formulations with 10 mg/mL EDTA and 20 mg/mL ethanol, the presence of 1 mg/mL Tween-80 (3-1, filled triangles) had a stabilizing effect over the same formulation without Tween-80 (3-2, open squares). Glutamate buffer (3-3, open diamonds) provided more stability compared to acetate buffer (3-2, open squares). The data for PYY₃₋₃₆ purity show that the predominant species remaining in solution has the same retention time by HPLC as native PYY₃₋₃₆, consistent with loss of peptide due to aggregation. The precipitate consisted predominantly of PYY₃₋₃₆ monomer, showing that the loss in PYY₃₋₃₆ upon subjection to thermal and atomization stresses is due to a hydrophobic (e.g., non-covalent) aggregation.

PYY₃₋₃₆ formulations as potentially subject to hydrophobic, e.g., non-covalent, aggregation upon exposure to elevated temperatures combined with the stress of thrice daily spraying. Under certain conditions, the presence of Tween-80 ameliorates this. Also, the data show that glutamate may be a preferred buffer system with respect to stability compared to acetate.

### Example 9

### Thermal Stability and Atomization Stress Stability for Various PYY3-36 Formulations

The objective of this study was to examine stability against thermal and atomization stresses for PYY₃₋₃₆ formulations containing ethanol, EDTA, and Tween 80 as potential permeation enhancers for PYY₃₋₃₆. In this example, acetate buffer was tested from a pH range from pH 3.8 to 4.4, and chlorobutanol was used as a preservative to allow for multi-use.

The methodologies employed in this example were the same as described in Example 8 above. As in Example 8, herein vials were stored at 30° C./65% relative humidity between all sprays (during the day as well as overnight), vials were sprayed three times per day (TID) for 10 days, and HPLC was utilized to determine PYY₃₋₃₆ content in the spray (last spray of the day) at day=0, 5 and 10 of spraying.

The various formulations tested in this example are described in Table 10. All samples contained 6 mg/mL PYY_{3-36, 10} mM acetate buffer (acetic acid/sodium acetate buffer system) and 5 mg/mL chlorobutanol (CB). Samples 4-1 through 4-8 contained 10 mg/mL EDTA, 20 mg/mL ethanol, and either 0 or 1 mg/mL Tween 80, and the pH was varied from 3.8 to 4.4. For comparison, the last sample, 4-9, contained 45 mg/mL methyl-beta-cyclodextrin, 1 mg/mL DDPC, and 1 mg/mL EDTA, pH 4.0. The latter formulation was described previously (U.S. patent application Ser. No. 10/768,288, Quay et al. “Compositions and methods for enhanced mucosal delivery of Y2 receptor-binding peptides and methods for treating and preventing obesity”).

TABLE 10
Description of Formulations Testing in Example 9

| | | | | | Tween | |
|---|---|---|---|---|---|---|
| | Me-β-CD | DDPC | EDTA | Ethanol | 80 | |
| Group # | (mg/mL) | (mg/mL) | (mg/mL) | (mg/mL) | (mg/mL) | pH |
| 4-1 | 0 | 0 | 10 | 20 | 1 | 3.8 |
| 4-2 | 0 | 0 | 10 | 20 | 0 | 3.8 |
| 4-3 | 0 | 0 | 10 | 20 | 1 | 4.0 |
| 4-4 | 0 | 0 | 10 | 20 | 0 | 4.0 |
| 4-5 | 0 | 0 | 10 | 20 | 1 | 4.2 |
| 4-6 | 0 | 0 | 10 | 20 | 0 | 4.2 |
| 4-7 | 0 | 0 | 10 | 20 | 1 | 4.4 |
| 4-8 | 0 | 0 | 10 | 20 | 0 | 4.4 |
| 4-9 | 45 | 1 | 1 | 0 | 0 | 4.0 |

Abbreviations:
Me-β-CD = methyl-beta-cycldoextrin
EDTA = disodium edentate
DDPC = L-α-phosphatidylcholine didecanoyl.

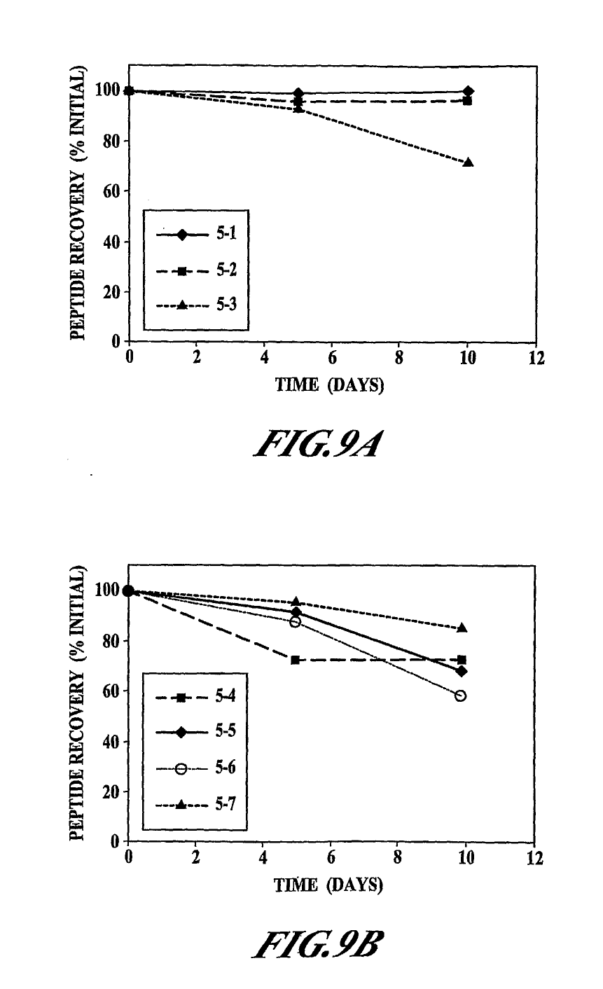

HPLC data for peptide content at day=0, day=5 and day=10 of the exposure to elevated temperature and atomization stress of thrice daily spraying are depicted in FIG. 8.

The data show that the presence of 1 mg/mL Tween-80 improves PYY₃₋₃₆ stability after combined thermal and atomization stresses (compare sample 4-1 and 4-2 (filled and open diamonds, respectively); compare sample 4-3 and 4-4 (filled and open squares, respectively); compare sample 4-5 and 4-6 (filled and open circles, respectively); and compare sample 4-7 and 4-8 (filled and open triangles, respectively)). There was also a trend for improved stability as the pH was lowered from 4.4 to 3.8. The sample at pH 3.8 containing 1 mg/mL Tween-80 (4-1), exhibited nearly the same stability as that for sample 4-9.

Presence of 1 mg/mL Tween-80 provided stabilization towards thermal and spraying stresses for PYY₃₋₃₆ formulations containing 10 mg/mL EDTA and 20 mg/mL ethanol. Stability was improved as the pH was lowered from 4.4 to 3.8.

### Example 10

### Thermal Stability and Atomization Stress Stability for PYY3-36 Formulations

The objective of this study was to examine stability against thermal and atomization stresses for PYY₃₋₃₆ formulations containing ethanol, EDTA, and Tween 80 as potential permeation enhancers for PYY₃₋₃₆. In this example, the buffer was either acetate or glutamate, the pH was 4.0, and the level of Tween-80 was varied from 0 to 50 mg/mL. Chlorobutanol was added as a preservative. Methods employed in this example were described in Example 8. Vials were stored at 30° C./65% relative humidity between all sprays, vials were sprayed TID for 10 days, and HPLC was utilized to determine PYY₃₋₃₆ content in the spray (last spray of the day) at day=0, 5, and 10 of spraying.

The various formulations tested in this example are described in Table 11. All samples contained 6 mg/mL PYY₃₋₃₆ and 5 mg/mL chlorobutanol (CB). Samples 5-1 through 5-13 contained 1-10 mg/mL EDTA, 10-20 mg/mL ethanol, 0-50 mg/mL Tween-80, either 10 mM acetate buffer/pH 5 or 10 mM glutamate buffer/pH 4. For comparison, the last sample, 5-14, contained 45 mg/mL methyl-beta-cyclodextrin, 1 mg/mL DDPC, and 1 mg/mL EDTA, pH 4.0.

TABLE 11
Description of Formulations Tested in Example 10

| | Me- | | | | | Gluta- | |
|---|---|---|---|---|---|---|---|
| | β- | | | Etha- | Tween | mate | Acetate |
| Group | CD | DDPC | EDTA | nol | 80 | Buffer | Buffer |
| # | (mg/mL) | | | | | mM | |
| 5-1 | 0 | 0 | 1 | 10 | 0 | 0 | 10 |
| 5-2 | 0 | 0 | 1 | 10 | 1 | 0 | 10 |
| 5-3 | 0 | 0 | 1 | 10 | 1 | 10 | 0 |
| 5-4 | 0 | 0 | 10 | 20 | 0 | 0 | 10 |
| 5-5 | 0 | 0 | 10 | 20 | 1 | 0 | 10 |
| 5-6 | 0 | 0 | 10 | 20 | 10 | 0 | 10 |
| 5-7 | 0 | 0 | 10 | 20 | 50 | 0 | 10 |
| 5-8 | 0 | 0 | 10 | 20 | 0 | 10 | 0 |
| 5-9 | 0 | 0 | 10 | 20 | 1 | 10 | 0 |
| 5-10 | 0 | 0 | 10 | 20 | 10 | 10 | 0 |
| 5-11 | 0 | 0 | 10 | 20 | 50 | 10 | 0 |
| 5-12 | 0 | 0 | 1 | 20 | 1 | 0 | 10 |
| 5-13 | 0 | 0 | 1 | 20 | 1 | 10 | 0 |
| 5-14 | 45 | 1 | 1 | 0 | 0 | 0 | 10 |

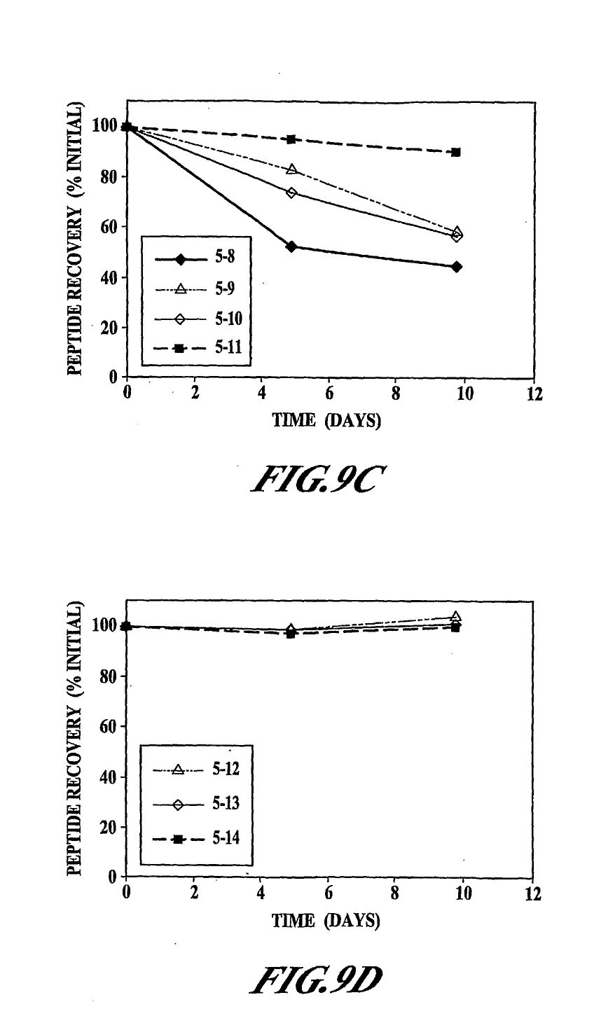

FIG. 9A shows the stability data for samples 5-1, 5-2, and 5-3. Comparison of 5-1 and 5-2 reveals that addition of 1 mg/mL Tween 80 did not have an improved effect since excellent stability was achieved for both samples under the conditions tested (e.g., 1 mg/mL EDTA and 10 mg/mL ethanol, pH 4). Comparison of sample 5-2 to 5-3 revealed a slightly lower stability observed for glutamate buffer v. acetate buffer under the conditions tested.

FIG. 9B depicts the stability data for 5-4, 5-5, 5-6 and 5-7. All these samples contained 10 mg/mL EDTA, 20 mg/mL ethanol, and 10 mM acetate buffer/pH 4.0. This series of samples had varying levels of Tween 80, namely from 0 to 50 mg/mL. In general, the stability observed under the conditions in FIG. 9B was slightly lower compared to the samples in FIG. 9A. The highest stability was observed for sample 5-7 which contained the highest level of Tween 80 (50 mg/mL) in this series.

FIG. 9C presents the effect of thermal and atomization stress for samples 5-4, 5-5, 5-6 and 5-7. All these samples contained 10 mg/mL EDTA, 20 mg/mL ethanol, and 10 mM glutamate buffer/pH 4.0. This series of samples had varying levels of Tween 80, namely from 0 to 50 mg/mL. In general, the stability observed under the conditions in FIG. 9C was slightly lower compared to the samples in FIG. 9A and FIG. 9B. The highest stability was observed for sample 5-11 which contained the highest level of Tween 80 (50 mg/mL) in this series.

Finally, the stability data for samples 5-12, 5-13 and 5-14 are illustrated in FIG. 9D. All three samples show excellent stability, namely, there is no substantial change in peptide recovery over the 10 days of exposure to thermal and atomization stress.

1-93. (canceled)

94. A pharmaceutical formulation for enhancing mucosal delivery of a Y2 receptor binding peptide to a mammal, wherein the formulation comprises a therapeutically effective amount of the peptide, a water-miscible polar organic solvent and a chelating agent for cations.

95. The pharmaceutical formulation of claim 94, wherein the Y2 receptor binding peptide is PYY or a functional analog thereof.

96. The formulation of claim 95, wherein PYY is PYY(3-36), the water-miscible polar organic solvent is ethanol, and the chelating agent for cations is EDTA.

97. The formulation of claim 96, wherein ethanol is at a formula concentration of about 1% (v/v) or greater.

98. The formulation of claim 96, wherein ethanol is at a formula concentration of about 2% (v/v) or greater.

99. The formulation of claim 96, wherein ethanol is at a formula concentration of about 10% (v/v) or greater.

100. The formulation of claim 96, wherein EDTA is at a concentration of at least about 1 mg/ml in the formulation.

101. The formulation of claim 96, wherein EDTA is at a concentration of at least about 2 mg/ml in the formulation.

102. The formulation of claim 96, wherein EDTA is at a concentration of at least about 10 mg/ml in the formulation.

103. The formulation of claim 96, further comprising a surface-acting agent.

104. The formulation of claim 103, wherein the surface-acting agent is Tween-80.

105. The formulation of claim 104, wherein Tween-80 is present at 50 mg/ml or lower in the formulation.

106. The formulation of claim 104, wherein Tween-80 is present at 10 mg/ml or lower in the formulation.

107. The formulation of claim 104, wherein Tween-80 is present at 1 mg/ml or lower in the formulation.

108. The formulation of claim 96, further comprising a buffer salt.

109. The formulation of claim 103, further comprising a buffer salt.

110. The formulation of claim 108, wherein the buffer salt is acetate or glutamate.

111. The formulation of claim 109, wherein the buffer salt is acetate or glutamate.

112. The formulation of claim 110, wherein the buffer salt is glutamate.

113. The formulation of claim 111, wherein the buffer salt is glutamate.

114. The formulation of claim 96, wherein the formulation has a pH of about 5.0 or less.

115. The formulation of claim 96, wherein the formulation has a pH of about 4.4 or less.

116. The formulation of claim 96, wherein the formulation has a pH of about 4.0 or less.

117. The formulation of claim 96, wherein the formulation has a pH of about 3.8 or less.

118. The formulation of claim 96, further comprising a preservative.

119. The formulation of claim 118, wherein the preservative is chlorobutanol or benzalkonium chloride.

120. The formulation of claim 96, wherein administration of the formulation by contact to a monolayer of mucosal cells results in a measured Papp of about 2-fold or greater compared to the Papp measured administering an isotonic solution devoid of permeation enhancers.

121. The formulation of claim 96, wherein administration of the formulation by contact to a monolayer of mucosal cells results in a measured Papp of about 5-fold or greater compared to the Papp measured administering an isotonic solution devoid of permeation enhancers.

122. The formulation of claim 96, wherein administration of the formulation by contact to a monolayer of mucosal cells results in a measured Papp of about 10-fold or greater compared to the Papp measured administering an isotonic solution devoid of permeation enhancers.

123. The formulation of claim 120, wherein the mucosal cells are bronchial epithelial cells.

124. The formulation of claim 121, wherein the mucosal cells are bronchial epithelial cells.

125. The formulation of claim 122, wherein the mucosal cells are bronchial epithelial cells.

126. The formulation of claim 96, wherein administration of the formulation intranasally in a mammal results in a measured AUCₗₐₛₜ of about 2-fold or greater compared to the AUCₗₐₛₜ measured for intranasal administration of an isotonic saline solution devoid of permeation enhancers.

127. The formulation of claim 96, wherein administration of the formulation intranasally in a mammal results in a measured AUCₗₐₛₜ of about 5-fold or greater compared to the AUCₗₐₛₜ measured for intranasal administration of an isotonic saline solution devoid of permeation enhancers.

128. The formulation of claim 96, wherein administration of the formulation intranasally in a mammal results in a measured AUCₗₐₛₜ of about 10-fold or greater compared to the AUCₗₐₛₜ measured for intranasal administration of an isotonic saline solution devoid of permeation enhancers.

129. The formulation of claim 96, wherein administration of the formulation intranasally in a mammal results in a measured AUCₗₐₛₜ of about 20-fold or greater compared to the AUCₗₐₛₜ measured for intranasal administration of an isotonic saline solution devoid of permeation enhancers.

130. The pharmaceutical formulation of claim 96, wherein the formulation comprises a therapeutically effective amount of PYY, about 2% (v/v) ethanol, about 10 mg/ml EDTA, about 1% Tween-80, and a pH of about 4.0.

131. The formulation of claim 130, further comprised of a preservative, wherein the preservative is chlorobutanol or benzalkonium chloride.

132. The formulation of claim 131, further comprising a buffer salt, wherein the buffer salt is acetate or glutamate.

133. The formulation of claim 132, wherein the buffer salt is glutamate.

134. A PYY dosage form suitable for multi-use administration comprising a sealed bottle containing an aqueous pharmaceutical formulation, wherein the formulation comprises a therapeutically effective amount of PYY, a water-miscible polar organic solvent and a chelating agent for cations, and wherein such PYY dosage form exhibits at least 90% PYY recovery after storage for at least 10 days at 5° C.

135. The PYY dosage form of claim 134, having greater than about 90% recovery of PYY after at least six months at 5° C. storage.

136. The PYY dosage form of claim 134, having greater than about 90% recovery of PYY after one year at 5° C. storage.

137. The PYY dosage form of claim 134, having greater than about 90% recovery of PYY after two years at 5° C. storage.

138. The PYY dosage form of claim 134, wherein the bottle further comprises an actuator effective for intranasal administration of the formulation, and wherein such dosage form exhibits at least 90% PYY recovery after storage as used for greater than about five days.

139. The PYY dosage form of claim 138, wherein the administration is thrice-daily sprays.

140. The PYY dosage form of claim 139, having greater than about 90% recovery of PYY at 30° C./65% relative humidity between all sprays.

141. The PYY dosage form of claim 134, further comprising a buffer having a net single ionogenic moiety with a pKa within two pH units of the pH of the formulation.

142. The PYY dosage form of claim 138, further comprising a buffer having a net single ionogenic moiety with a pKa within two pH units of the pH of the formulation.

143. The PYY dosage form of claim 141, wherein said buffer has a net single ionogenic moiety with a pKa within one pH unit of the pH of the formulation.

144. The PYY dosage form of claim 142, wherein said buffer has a net single ionogenic moiety with a pKa within one pH unit of the pH of the formulation.

145. The PYY dosage form of claim 141, wherein said buffer is selected from the list consisting of glutamate, acetate, glycine, histidine, arginine, lysine, methionine, lactate, formate, and glycolate.

146. The PYY dosage form of claim 142, wherein said buffer is selected from the list consisting of glutamate, acetate, glycine, histidine, arginine, lysine, methionine, lactate, formate, and glycolate.

147. The PYY dosage form of claim 146, wherein the buffer is glutamate or acetate.

148. The PYY dosage form of claim 146, wherein the pH is about 5.0 or less.

149. The PYY dosage form of claim 146, wherein the pH is about 4.4 or less.

150. The PYY dosage form of claim 146, wherein the pH is about 4.0 or less.

151. The PYY dosage form of claim 146, wherein the pH is about 3.8 or less.

152. The PYY dosage form of claim 134, wherein PYY is PYY(3-36).

153. The PYY dosage form of claim 138, wherein PYY is PYY(3-36).

154. The PYY dosage form of claim 152, wherein the concentration of PYY is at least about 20 μg/ml.

155. The PYY dosage form of claim 152, wherein the concentration of PYY is at least about 100 μg/ml.

156. The PYY dosage form of claim 152, wherein the concentration of PYY is at least about 200 μg/ml.

157. The PYY dosage form of claim 152, wherein the concentration of PYY is at least about 1 mg/ml or greater.

158. The PYY dosage form of claim 152, wherein the concentration of PYY is at least about 2 mg/ml or greater.

159. The PYY dosage form of claim 152, wherein the concentration of PYY is at least about 6 mg/ml or greater.

160. The PYY dosage form of claim 152, wherein the concentration of PYY is at least about 10 mg/ml or greater.

161. The PYY dosage form of claim 134, wherein said dosage form is suitable for intra-nasal administration to achieve a dose of from about 2 μg to about 1000 μg of said PYY.

162. The PYY dosage form of claim 134, wherein said dosage form is suitable for intra-nasal administration to achieve a dose of from about 100 μg to about 600 μg of said PYY.

163. The PYY dosage form of claim 134, wherein the water-miscible polar organic solvent is ethanol and the chelating agent for cations is EDTA.

164. The PYY dosage form of claim 163, wherein ethanol is at a formula concentration of at least about 0.1% (v/v).

165. The PYY dosage form of claim 163, wherein ethanol is at a formula concentration of at least about 1% (v/v).

166. The PYY dosage form of claim 163, wherein ethanol is at a formula concentration of at least about 10% (v/v).

167. The PYY dosage form of claim 163, wherein EDTA is at a concentration of at least about 1 mg/ml in the formulation.

168. The PYY dosage form of claim 163, wherein EDTA is at a concentration of at least about 10 mg/ml in the formulation.

169. The PYY dosage form of claim 163, wherein EDTA is at a concentration of at least about 50 mg/ml in the formulation.

170. The PYY dosage form of claim 163, further comprising a surface-acting agent.

171. The PYY dosage form of claim 163, wherein the surface-acting agent is Tween-80.

172. The PYY dosage form of claim 171, wherein Tween-80 is present at least about 1 mg/ml in the formulation.

173. The PYY dosage form of claim 171, wherein Tween-80 is present at least about 10 mg/ml in the formulation.

174. The PYY dosage form of claim 171, wherein Tween-80 is present at least about 50 mg/ml in the formulation.

175. The PYY dosage form of claim 163, further comprising a preservative.

176. The PYY dosage form of claim 175, wherein the preservative is chlorobutanol or benzalkonium chloride.
